(19) United States
(12) Patent Application Publication
(10) Pub. No.: US 2023/0352122 A1
(43) Pub. Date: Nov. 2, 2023

(54) HYDROCARBON FLUID PROPERTIES PREDICTION USING MACHINE-LEARNING-BASED MODELS

(71) Applicants: Abu Dhabi National Oil Company, Abu Dhabi (AE); Abu Dhabi Company for Onshore Petroleum Operations Limited, Abu Dhabi (AE)

(72) Inventors: Arwa Ahmed MAWLOD, Abu Dhabi (AE); Richard MOHAN, Abu Dhabi (AE); Kassem GHORAYEB, Abu Dhabi (AE); Hussein MUSTAPHA, Abu Dhabi (AE)

(21) Appl. No.: 18/041,093

(22) PCT Filed: Aug. 13, 2021
(86) PCT No.: PCT/IB2021/057475

§ 371 Date: Feb. 9, 2023

(30) Foreign Application Priority Data
Aug. 19, 2020 (EP) 20191806.7

(51) Int. Cl. G16C 20/30; G01N 33/28; G16C 20/70; G06N 20/00

(52) U.S. Cl. CPC G16C 20/30; G01N 33/2823; G16C 20/70; G06N 20/00

(57) ABSTRACT

A computer-implemented method for predicting hydrocarbon fluid properties comprises the step of receiving an incomplete set of pressure-volume-temperature (PVT) data for hydrocarbon fluid samples from a PVT data base; reading the incomplete set of PVT data; transforming the incomplete set of PVT data into a unified data structure; selecting items of the PVT data from the incomplete set of PVT data; processing the selected items of the PVT data to identify a plurality of correlations in the selected items of the PVT data based on one or more of the fluid properties of the hydrocarbon fluid samples; clustering, using at least one of a plurality of clustering schemes, the selected items of the PVT data into a plurality of clusters; and performing machine learning on the plurality of clusters to predict missing fluid properties in the incomplete set of PVT data to obtain a complete set of PVT data.

This application is a national phase of International Application No. PCT/IB2021/057475, filed Aug. 13, 2021, which claims priority to European Patent Application No. 20191806.7, filed Aug. 19, 2020, each of which is hereby incorporated herein by reference in its entirety.

### FIELD OF THE INVENTION

The present invention relates to a computer-implemented method and system for predicting hydrocarbon fluid properties using machine-learning-based models. The present invention further relates to a computer-implemented method and system for generating equations of state (EoS) for a plurality of hydrocarbon fluids using machine-learning-based models.

### BACKGROUND OF THE INVENTION

Petroleum consists of a complex mixture of hydrocarbons of various molecular weights, plus other organic compounds. The exact molecular composition of petroleum varies widely from formation to formation. The proportion of hydrocarbons in the mixture is highly variable and ranges from as much as 97% by weight in the lighter oils to as little as 50% in the heavier oils and bitumens. The hydrocarbons in petroleum are mostly alkanes (linear or branched), cycloalkanes, aromatic hydrocarbons, or more complicated chemicals like asphaltenes. The other organic compounds in petroleum typically contain carbon dioxide (CO2), nitrogen, oxygen, and sulfur, and trace amounts of metals such as iron, nickel, copper, and vanadium.

Knowledge of hydrocarbon fluid properties are of importance in the oil and gas industry. The fluid properties are essential for calculating the amount of the hydrocarbons initially in place, for reservoir simulation and production forecasting as well as for well, completion, pipeline and surface facility design. For measuring hydrocarbon fluid properties, typically, the pressure-volume-temperature (PVT) properties are measured as a function of pressure. Therefore, PVT data for hydrocarbon fluid samples is necessary. This PVT data may be acquired at different location of the production system: e.g., well bottom hole, well tubing head, and at the outlet of the last separator stage. Once acquired, this PVT data is sent to the laboratory for analysis where the fluid properties are measured. Nevertheless, regardless of the number of PVT data for hydrocarbon fluid samples that are acquired and the laboratory measurements that are performed, a thermodynamic model is typically used, such as an equation of state (EoS) model that represents the phase behavior of the petroleum fluid in the reservoir and is used to predict the hydrocarbon fluid properties under the expected range of pressure and temperature covering the life of the reservoir and the whole production system. Once the EoS model is defined, the EoS model can be used to compute a wide array of properties of the petroleum fluid of the reservoir, such as gas-oil ratio (GOR) or condensate-gas ratio (CGR), density of each phase, volumetric factors and compressibility, and heat capacity and saturation pressure (bubble or dew point). Thus, the EoS model can be solved to obtain saturation pressure at a given temperature. Moreover, GOR, CGR, phase densities, and volumetric factors are byproducts of the EoS model. Other properties, such as heat capacity or viscosity, can also be derived in conjunction with the information regarding fluid composition. Furthermore, the EoS model can be extended with other reservoir evaluation techniques for compositional simulation of flow and production behavior of the petroleum fluid of the reservoir, as is known in the art.

To validate a typical EoS model from laboratory measurements, a minimum set of measurements are required e.g. compositional properties (mole fractions of the components (e.g. N2, H2S, CO2, C1, C2, C3, C4, C5, C7, C8, etc.) and pseudo-components (e.g. C7+, C12+, C20+ and C36+) as well as the molecular weight of the pseudo-components), constant composition expansion (CCE), constant volume depletion (CVD), differential liberation (DL) and multi-stage separator (MSS).

Building machine-learning-based models based on the PVT data enables the capture of trends and prediction of fluid models in a very heterogeneous thermodynamic system exhibiting highly non-linear behaviors. The PVT data for hydrocarbon fluid samples stored in an oil company database are often not complete and might be missing properties, both black oil and compositional properties. Therefore, it is required to have a clean and structured PVT database complete with all required properties. In the presence of a large set of the PVT data for hydrocarbon fluid samples, some of these PVT data may lack part of the fluid properties as they may not have been measured in the laboratory. In some scenarios, the compositional properties in the database are absent as no representative PVT data could be obtained. In this case, the list of measurements is restricted to some properties obtained at stock tank conditions. A large set of existing PVT data may be lacking the PVT granularity of the heavy end of the hydrocarbon spectrum (e.g. they may be restricted to C7+). Companies with multiple fields and reservoirs may have an extensive set of the EoS models built using different PVT data (with their corresponding laboratory analysis). When new PVT data is acquired for a new field or from a specific region of an existing field, is it necessary to measure similarities with the existing PVT data of hydrocarbon fluid samples to either map the sample to an existing fluid model or intelligently predict a new one.

Predicting the PVT data properties using machine learning or artificial intelligence is known in the art. With respect to predicting compositional properties from other compositional properties, Wang et al. (Wang, K., Zuo, Y. and Jalali, Y., Schlumberger Technology Corp, 2017. Prediction of Fluid Composition and/or Phase Behavior. U.S. patent application Ser. No. 15/193,519) propose and test machine learning algorithms to predict and/or calculate components mole fractions (CO2, C1, C2, C3, C4, C5 and C6+) as well as C6+ molecular weight from components weight fractions. Wang et al. describe two workflows. A first workflow starts from weight fractions of CO2, C1, C2, C3, C4, C5 and C6+, predicts using machine learning the mole fraction of C6+ and then calculates, using pertinent equations relating molar weight to mole fractions and mass fractions, the molecular weight of C6+ followed by calculating, using pertinent equations relating mole fractions to weight fractions, the mole fractions of CO2, C1, C2, C3, C4, C5. The other workflow starts from weight fractions of CO2, C1, C2, C3, C4, C5 and C6+ and predicts using machine learning the mole fractions of CO2, C1, C2, C3, C4, C5 and C6+.

The predicting of the PVT data properties is further described in Almashan, Meshal & Narusue, Yoshiaki & Morikawa, Hiroyuki. (2019). Estimating PVT Properties of Crude Oil Systems Based on a Boosted Decision Tree Regression Modelling Scheme with K-Means Clustering. Asia Pacific Oil & Gas Conference and Exhibition, 25 Oct. 2019, 10.2118/196453-MS. The use of modelling approaches using machine learning to predict the PVT data properties is disclosed. The model proposed in the document is used for predicting the bubble point pressure (Pb) and the oil formation volume factor at bubble point pressure (Bob) as a function of oil and gas specific gravity, solution gas-oil ratio, and reservoir temperature by a boosted decision tree regression (BDTR) predictive modelling scheme.

Oloso, Munirudeen & Hassan, M. G. & Bader-El-Den, Mohamed & Buick, James. (2017). Hybrid Functional Networks for Oil Reservoir PVT Characterisation. Expert Systems with Applications. 12 Jun. 2017. 87. 10.1016/j.eswa.2017.06.014 discloses a method and system for prediction of crude oil PVT data. The method and system are used for prediction of a bubblepoint pressure (Pb) and an oil formation volume factor at bubblepoint pressure (Bob). The modelling is done using K-means clustering and functional networks comprising machine learning algorithms and neural networks.

Elsebakhi, Emad. (2009). Data mining in forecasting PVT correlations of crude oil systems based on Type1 fuzzy logic inference systems. Computers & Geosciences. 35. 1817-1826. 10.1016/j.cageo.2007.10.016 describes the use of adaptive neuro-fuzzy inference systems for a prediction of PVT-properties. The focus of the El-Sebakhy publication lies in the comparison of different approaches for the prediction of PVT-properties.

As discussed above, oil companies with multiple fields and reservoirs may have multiple ones of the EoS models built using different PVT data for hydrocarbon fluid samples (with their corresponding laboratory analysis). One or more PVT data sets may be used to build and calibrate the EoS model. These EoS models are typically built to model volumetric properties and phase behavior of one or more reservoirs or even a region in one reservoir in a specific field. The EoS models are also necessary to build dynamic models of the subsurface reservoirs, and these EoS models are the drivers in understanding reservoir performance and optimizing field development plans. The representativity of the reservoir with accurate fluid models is a big challenge, costly job, and it requires intensive and rigorous analysis. Multiple challenges are encountered through the process. Different known EoS models are built by different experts without coordination between these experts. The known EoS models are then tuned using different tuning parameters due to the non-uniqueness of the tuning process. Furthermore, different component and pseudo-components grouping/lumping schemes are applied by different experts as, again, the grouping/lumping is not a unique process, by nature.

### SUMMARY OF THE INVENTION

It is an object of the present invention to provide a computer-implemented method and system for predicting hydrocarbon fluid properties using machine-learning-based models. It is further an object of the present invention to provide an improved computer-implemented method and system for generating equations of state (EoS) for a plurality of hydrocarbon fluids from the predicted hydrocarbon fluid properties.

In view of the state of the known technology and in accordance with a first aspect of the present invention, a computer-implemented method for predicting hydrocarbon fluid properties using machine-learning-based models is described in this document. This method comprises the method steps of: receiving an incomplete set of pressure-volume-temperature (PVT) data for hydrocarbon fluid samples from a PVT data base, wherein the incomplete set of PVT data comprises black oil properties and compositional properties; reading the incomplete set of PVT data by a reader module; transforming the incomplete set of PVT data into a unified data structure by the reader module, wherein the unified data structure is used for storing items of data input from different sources in a unified way; selecting items of the PVT data from the transformed incomplete set of PVT data by the reader module; processing the selected items of the PVT data by a correlating module to identify a plurality of correlations in the selected items of the PVT data based on one or more of the fluid properties of the hydrocarbon fluid samples; clustering, using of at least one of a plurality of clustering schemes, the selected items of the PVT data into a plurality of clusters by a clustering module; and performing machine learning by a machine learning module on ones of the plurality of clusters to predict missing fluid properties in the transformed incomplete set of PVT data and thus to obtain a complete set of PVT data. The complete set of PVT data comprises the black oil properties and compositional properties of the incomplete set of PVT data, wherein the complete set of PVT data comprises the black oil properties and compositional properties of the incomplete set of PVT data and further comprises predicted items of data for the black oil properties and predicted compositional properties.

The computer-implemented method in accordance with the first aspect of the present invention provides a systematic methodology to complete PVT data for properties for hydrocarbon fluid in a consistent manner.

In another aspect, the step of performing machine learning by the machine learning module of the computer-implemented method further comprises the step of predicting of fluid properties for incomplete sets of PVT data for the hydrocarbon fluid samples.

In another aspect, the computer-implemented method comprises the step of plotting the machine learning predictions by the machine learning module.

In another aspect, the computer-implemented method comprises the step of comparing the identified plurality of correlation results with the machine learning predictions.

In another aspect, the step of clustering of the computer-implemented method further comprises the step of completing fluid composition including C12+, C20+ and C36+ mole fraction and molecular weight and/or completing black oil properties, including, in the following order: solution gas oil ratio (GOR), BO@Psat, and saturation pressure (Psat).

In view of the state of the known technology and in accordance with a second aspect of the present invention, a system for predicting hydrocarbon fluid properties using machine-learning-based models is disclosed. The system comprises a pressure-volume-temperature (PVT) data base, a reader module, a correlating module, a clustering module, and a machine learning module. The pressure-volume-temperature (PVT) data base provides an incomplete set of pressure-volume-temperature (PVT) data for hydrocarbon fluid samples, wherein the incomplete set of PVT data comprises ones of black oil properties and compositional properties. The reader module reads in an incomplete set of PVT data, wherein the reader module is configured to transform the incomplete set of PVT data into a unified data structure, and wherein the unified data structure is used for storing items of data input from different sources in a unified way, and wherein the reader module is configured to select items of the PVT data from the incomplete set of PVT data using exploratory data analysis (EDA). The correlating module for processes the selected items of the transformed incomplete set of PVT data to identify a plurality of correlations in the selected items of the PVT data based on one or more of the fluid properties of the hydrocarbon fluid samples. The clustering module clusters, using of at least one of a plurality of clustering schemes, the selected items of the transformed incomplete set of PVT data into a plurality of clusters. The machine learning module performs machine learning on ones of the plurality of clusters to predict missing fluid properties in the incomplete set of PVT data and thus to obtain a complete set of PVT data, wherein the predicted complete set of PVT data comprises the black oil properties and compositional properties of the incomplete set of PVT data and further comprises the predicted items of data for the ones of the black oil properties and predicted compositional properties.

In view of the state of the known technology and in accordance with a third aspect of the present invention, a computer-implemented method for generating equations of state (EoS) for a plurality of hydrocarbon fluids from a predicted complete set of PVT data is disclosed. The method comprises the method steps of: delumping pressure-volume-temperature (PVT) data for hydrocarbon fluid samples from the predicted complete set of PVT data to one of a set of detailed fluid components, or to a common set of components and pseudo-components; lumping the PVT data from the predicted complete set of PVT data into a pre-defined set of components and pre-defined set of pseudo-components to generate a plurality of equation of state (EoS) models; generating for the PVT samples on the PVT data set an EoS model using a same set of tuning parameters and thereby generating an EoS fluid model fingerprint for the hydrocarbon fluid samples; and associating properties of the hydrocarbon fluid samples with the generated EoS fluid model fingerprint.

The computer-implemented method in accordance with the third aspect of the present invention leverages advancement in data sciences to unlock hidden information, patterns and relationships from massive volume and variety of reservoir hydrocarbon fluid data. The developed AI based algorithms can be used to consistently validate the hydrocarbon fluid data (also called PVT data), cluster the data and build machine learning based fluid models, i.e. equation of state (EoS), for different fields and reservoirs based on massive reservoir fluid information. The computer-implemented method in accordance with the second aspect of the present invention ensures reservoir models are managed with a high degree of confidence and accuracy with quality fluid data, maximizing returns while minimizing costs associated with EoS models.

In another aspect, the computer-implemented method comprises the step of training machine learning models to predict an EoS fluid model fingerprint for new hydrocarbon fluid samples.

In another aspect, the received or provided incomplete set of PVT data for hydrocarbon fluid samples comprises black oil properties and compositional properties. The black oil properties comprise at least one of reservoir temperature, solution gas-oil ratio, oil API gravity, gas gravity, dead oil viscosity, saturation pressure, saturated bubble point oil formation factor at saturation pressure, fluid density at reservoir conditions or any other black oil property, fluid compressibility at reservoir conditions, viscosity at reservoir conditions, fluid density at reservoir conditions or any other black oil property, and wherein the compositional properties comprise at least one of mole fractions of the components, in particular N2, H2S, CO2, C1, C2, C3, C4, C5, C7, C8, and pseud-components, in particular C7+, C12+, C20+ and C36+ or any other pseudo-component as well as the molecular weight of the pseudo-components.

In another aspect, the step of associating of the computer-implemented method further comprises the step of clustering the selected items of the PVT data into a plurality of clusters for performing machine learning on each one of the plurality of clusters.

In another aspect, the computer-implemented method comprises the step of comparing the results from clustering of the selected items of the PVT data with the plurality of equations of state (EoS) models and with heatmaps applying EoS models on the selected items of the PVT data.

In another aspect, the step of clustering of the selected items of the PVT data further comprises the step of identifying of clusters to which PVT data belong.

In view of the state of the known technology and in accordance with a fourth aspect of the present invention, a system for generating equations of state (EoS) for a plurality of hydrocarbon fluids from a predicted complete set of PVT data is disclosed. The system comprises a first module, a second module, a third module and a fourth module. The first module delumps pressure-volume-temperature (PVT) data for hydrocarbon fluid samples from the predicted complete set of PVT data to one of a set of detailed fluid components, or to a common set of components and pseudo-components. The second module lumps the PVT data for hydrocarbon fluid sample into a pre-defined set of components and pre-defined set of pseudo-components to generate a plurality of equation of state (EoS) models. The third module generates for the hydrocarbon fluid samples in a PVT data base an EoS model using a same set of tuning parameters and thereby generating an EoS fluid model fingerprint for the hydrocarbon fluid samples. The fourth module associates properties of the hydrocarbon fluid samples with the generated EoS fluid model fingerprint.

The present invention has a great impact on the way fluid modeling is performed. The present invention provides greater insights and better understanding of oil and gas fields and reservoirs. Furthermore, the results of the methods presented in the present invention can be scaled in very dynamic way to other fields in other regions. The present methods are based on a smart technology that adapts to the physical variation across fluid systems and perform fluid modeling with the highest confidence and accuracy. The technology is extendable and subject to integration with other subsurface domains as a smart fluid laboratory.

Also, other objects, features, aspects and advantages of the disclosed method will become apparent to those skilled in the art from the following detailed description.

### BRIEF DESCRIPTION OF THE DRAWINGS

The invention will now be described on the basis of figures. It will be understood that the embodiments and aspects of the invention described in the figures are only examples and do not limit the protective scope of the claims in any way. The invention is defined by the claims and their equivalents. It will be understood that features of one aspect or embodiment of the invention can be combined with a feature of a different aspect or aspects of other embodiments of the invention. The present invention becomes more obvious when reading the following detailed descriptions of some examples as part of the disclosure under consideration of the enclosed drawings. Referring now to the attached drawings which form a part of this disclosure:

FIG. 1A is a schematic illustration of Fields and Reservoirs and PVT data spread over the two reservoirs, in particular of three field illustrating two reservoirs.

FIG. 1B is a flow diagram of a computer-implemented method for predicting hydrocarbon fluid properties using machine-learning-based models in accordance with a first aspect of the present invention.

FIG. 1C is a flow diagram describing a chain algorithm for data completion in a compositional data and a black oil data.

FIG. 1D is an overview of a correlation of composition properties over a mole fraction C7+.

FIG. 1E is an overview of a correlation of composition properties over a molecular weight C7+.

FIG. 1F shows a flow diagram describing steps of a ML algorithm.

FIG. 2 is a schematic illustration of the overall view of the workflow diagram of the computer-implemented method for predicting hydrocarbon fluid properties using machine-learning-based models in accordance with the first aspect of the present invention.

FIG. 3 is a schematic illustration of a system for predicting hydrocarbon fluid properties using machine-learning-based models in accordance with a second aspect of the present invention.

FIG. 4 is a workflow diagram of the data reader module.

FIG. 5 is a workflow diagram of the clustering module.

FIG. 6 is a workflow diagram of the correlating module.

FIG. 7 is a workflow diagram of the machine learning module.

FIG. 8 is a table of data ingestion and preparation for machine learning.

FIG. 9 is a PVT Data set used in the implementation and validation process, in particular data statistics and black oil properties.

FIG. 10 is a PVT Data set used in the implementation and validation process, particular data statistics, compositional properties, and heavy fractions.

FIG. 11 is a PVT Data set used in the implementation and validation process, in particular data statistics, compositional properties, light and intermediate components.

FIGS. 12 and 13 are EDA Results applied to the PVT Data set used in the implementation and validation process, in particular FIG. 12 for black oil properties and FIG. 13 for black oil and compositional properties.

FIGS. 14 and 15 are EDA Results, in particular black oil properties, in particular shows FIG. 14 the worst correlations and FIG. 15 the best correlations. The properties included in the EDA of FIGS. 14 and 15 are illustrated in FIG. 12.

FIGS. 16A and 16B are EDA Results, in particular compositional properties, in particular shows FIG. 16A the worst correlations and FIG. 16B the best correlations. The properties included in the EDA of FIGS. 16A and 16B are illustrated in FIG. 13.

FIG. 17A is a flow diagram of a computer-implemented method for generating equations of state (EoS) for a plurality of hydrocarbon fluids in accordance with a second aspect of the present invention.

FIG. 17B shows a flow chart describing a workflow for training of a ML algorithm and saving models generated during the training of the ML algorithm.

FIG. 17C shows a workflow describing a method for clustering and saving the ML algorithms and ML models.

FIG. 18 is a schematic illustration of a system for generating equations of state (EoS) for a plurality of hydrocarbon fluids in accordance with a fourth aspect of the present invention.

FIG. 19 is an overview diagram of the hydrocarbon fluid properties and EoS prediction using machine learning subject of the present invention.

FIG. 20A is an overview of the machine-learning based EoS workflow.

FIG. 20B shows a table with a set of KPIs that can be used for a numerical evaluation of the EoS.

FIG. 21 illustrates the lumping scheme used in the process of creating the EoS model using regression.

FIG. 22 is a Heatmap resulting from Exploratory Data analysis (EDA) preceding ML to predict the EoS Parameters. TCRIT (C20P) and PCRIT (C20P) dependency on different BO and Compositional parameters. Results: Parameters to be used in ML are: Mole Fraction of C1, C6, C7+, C12+, C20+, C36+, MW of C7+, C12+, C36+, Psat, GOR, Bo@Psat, Density@Psat, API Gravity and Tres. Furthermore, a chain algorithm is used: PCRIT of C20P is added to inputs to predict TCRIT of C20P.

FIGS. 23A and 23B are an illustration of the predicted EoS parameters using ML when using Pcrit and Tcrit of C20P of C20P as regression parameters. The figures depicts Pcrit (FIG. 23A) and Tcrit (FIG. 23B) of C20P from ML vs. those obtained using conventional Regression.

FIG. 24 illustrates example results of machine-learning based EoS—Sample 1, Field F1, Reservoir R1. The figure shows actual data, initial guess of EoS model without any regression, Conventional regression based EoS and ML based EoS.

FIG. 25 illustrates other example results of machine-learning based EoS—Sample 2, Field F2, Reservoir R2. The figure shows actual data, initial guess of EoS model without any regression, Conventional regression based EoS and ML based EoS.

FIG. 26 illustrates other example results of machine-learning based EoS—Sample 3, Field F3, Reservoir R3. The figure shows actual data, initial guess of EoS model without any regression, Conventional regression based EoS and ML based EoS.

FIG. 27 illustrates machine-learning Based EoS—Error Analysis; Train+Test dataset. The EoS parameters resulting from ML are used in a PVT modeling tool to reproduce Laboratory experiments (CCE, DL and MSS). The figure shows the cumulative percentage error (EoS vs. Data) from conventional regression based EoS and ML based EoS.

FIG. 28 illustrates machine-learning Based EoS—Error Analysis; Train+Test dataset. The EoS parameters resulting from ML are used in a PVT modeling tool to reproduce Laboratory experiments (CCE, DL and MSS). The figure shows the cumulative percentage error (EoS vs. Data) from conventional regression based EoS and ML based EoS for a selected set of fluid properties resulting from the above-mentioned experiments.

### DETAILED DESCRIPTION OF THE INVENTION

Selected embodiments are now described with reference to the drawings. It is apparent from this disclosure to a person skilled in the art of generic fluid hydrocarbon fluid properties that the following description of embodiments is provided for illustrative purposes only and is not intended to limit the invention defined by the claims attached and their equivalents.

The following nomenclature is used in the present document:
MW=Molecular Weight
N_Samples=Number of PVT Samples (a total of 1700 PVT samples)
DETAILED COMPOSTION: detailed set of components=CO2, H2S, N2, C1, C2, C3, iC4, nC4, iC5, nC5, C6, C7, . . . , C35, C36+
EXPERIMENTS=Selected set of experiments. Set includes CCE, CVD, DL and MSS. CCE may be a good candidate as a proof of concept.
   Note: An entire experiment can be used. Alternatively, a selected set of properties from an experiment can be used. Example: Psat, Bo@Psat, GOR, API, etc.
SAMPLES=Selected set of PVT samples with their associated EXPERIMENTS. This may be the complete set of samples (N_Samples), all oil samples or the set validated oil samples used to build the EoS models. In this milestone, focus would be on the Validated Oil Samples.
EoS MODELS=Set of EoS models to be used in Tasks 2 and 3. The total number is ˜20.
TUNING PARAMETERS=Set of EoS parameters (P1, P2, P3, . . . , PN) to be tuned/modified, the range of uncertainty of these parameters and the associated weights.
FLUID PROPERTIES=PVT properties as set out below denote a multitude of hydrocarbon fluid properties under different temperatures and pressures, from the reservoir (at reservoir pressure and temperature) to the last separator stage; that is stock tank oil and gas at standard conditions. Properties are typically gathered under two categories:
   Compositional properties: These are mainly the mole fractions of the components (e.g. N2, H2S, CO2, C1, C2, C3, C4, C5, C7, C8, etc.) and pseud-components (e.g. C7+, C12+, C20+ and C36+ or any other pseudo-component) as well as the molecular weight of the pseudo-components. Note that the choice of pseudo-components used for the purpose of this project is not unique. The proposed algorithm work for any choice of pseudo-components.
   Black Oil Properties. Examples include, but not restricted to, properties presented in the below tables

| | |
|---|---|
| T_R | Reservoir Temperature |
| R_s | Solution gas-oil ratio |
| γ_API | Oil API gravity |
| γ_g | Gas gravity |
| μ_od | Dead oil viscosity |
| Psat | Saturation pressure |
| Bob @ Psat | Saturated bubble point oil formation factor at saturation |
| | pressure |
| ρ_psat | Fluid density at reservoir conditions |
| C_psat | Fluid compressibility at reservoir conditions |
| μ_psat | Viscosity at reservoir conditions |
| ρ_res | Fluid density at reservoir conditions |

FIELD=An accumulation, pool or group of pools of oil/gas in the subsurface. An oil/gas field comprises one or more reservoirs in a shape that will trap hydrocarbons and that are covered by an impermeable or sealing rock. Typically, industry professionals use the term with an implied assumption of large economic size.
RESERVOIR=A subsurface body of rock having sufficient porosity and permeability to store and transmit fluids. Sedimentary rocks are the most common reservoir rocks because they have more porosity than most igneous and metamorphic rocks and form under temperature conditions at which hydrocarbons can be preserved.

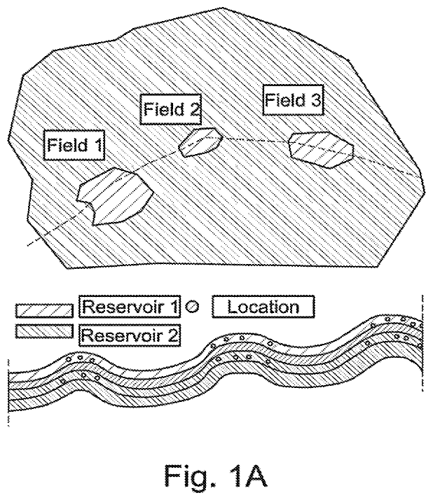

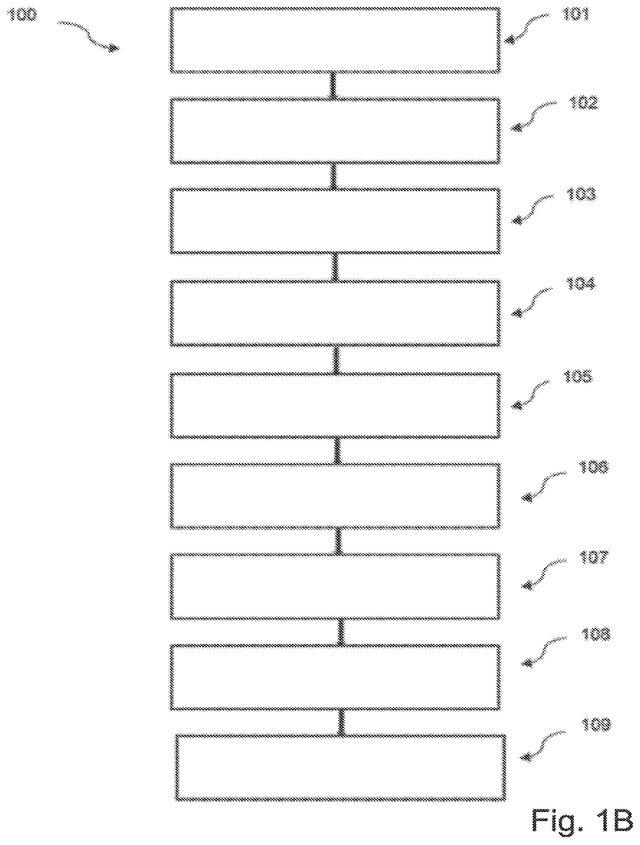

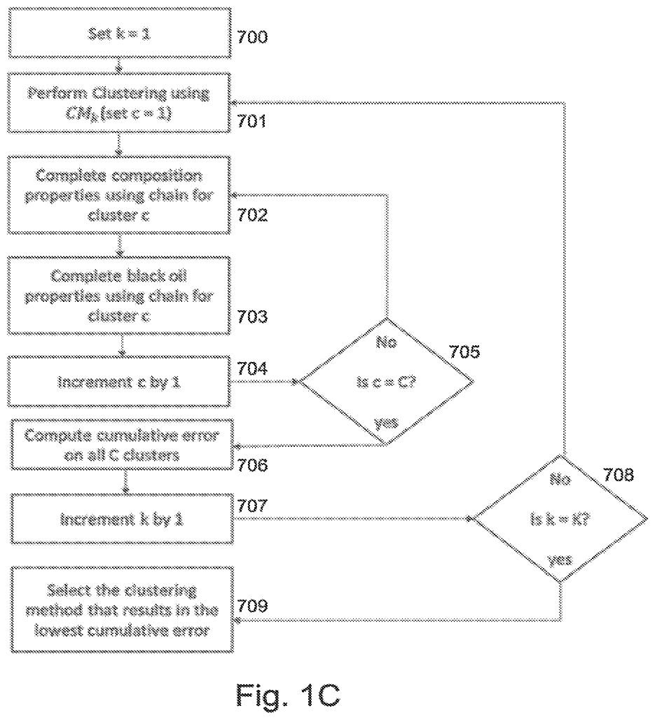

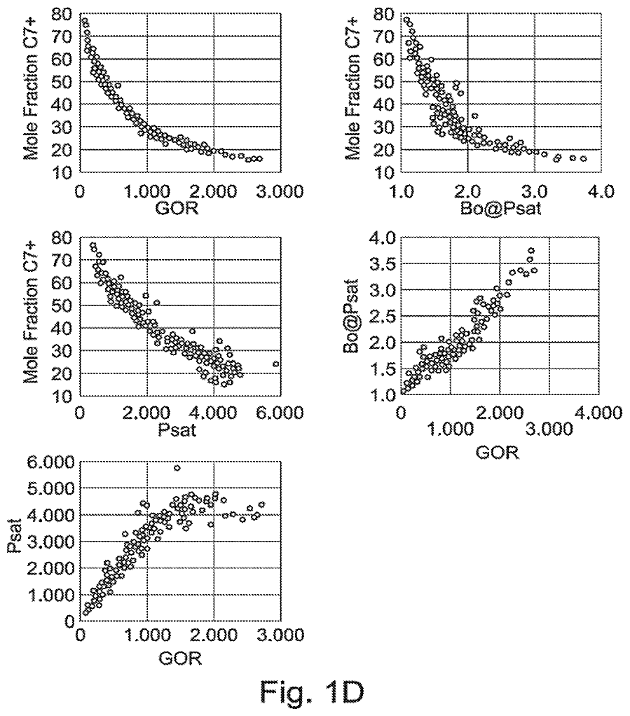

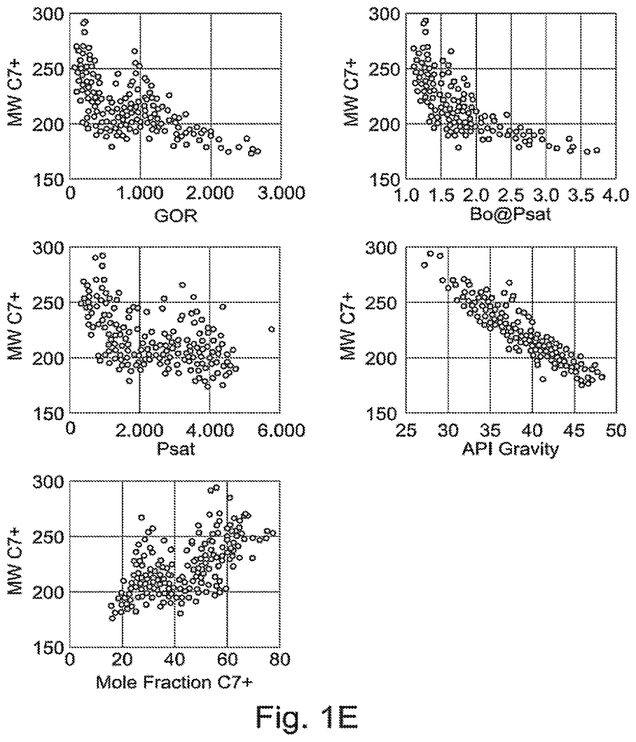

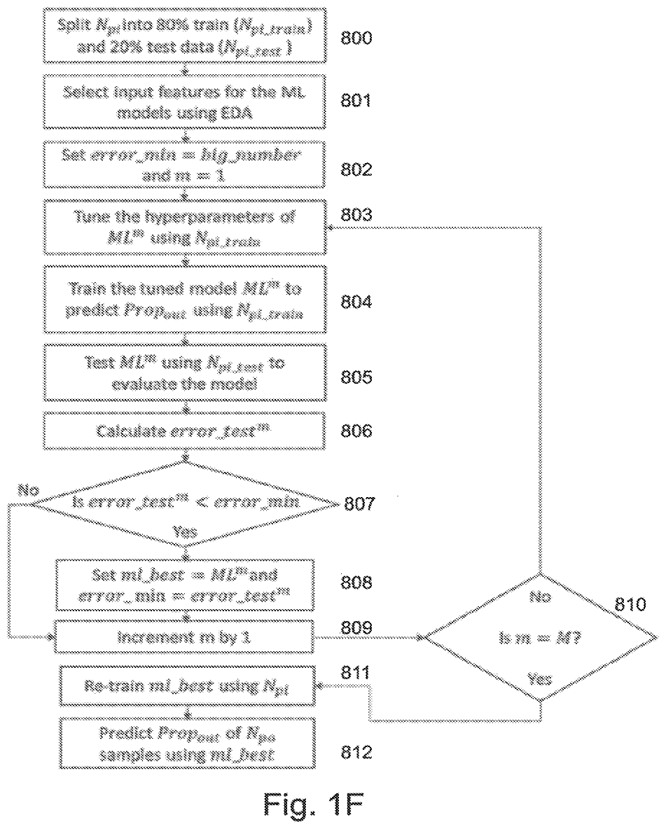

FIG. 1A is a schematic illustration of fields and reservoirs and PVT data spread over two reservoirs and illustrates three fields in two reservoirs.

FIG. 1B is a flow diagram of a computer-implemented method for predicting hydrocarbon fluid properties using machine-learning-based models in accordance with a first aspect of the present invention. The computer-implemented method 100 for predicting the hydrocarbon fluid properties using machine-learning-based models comprises the step of receiving 101 an incomplete set of pressure-volume-temperature (PVT) data for hydrocarbon fluid samples from a PVT data base 301 (shown in FIG. 3). The method 100 further comprises the step of reading 102 the incomplete set of the PVT data by a reader module 302. The method 100 comprises the step of transforming 103 the incomplete set of PVT data into a unified data structure by the reader module 302. The unified data structure is used for storing items of data input from different sources in a unified way in, for example, a central repository. The items of data can be input from tables, such as those found in Excel-files, exported from databases, such as a SQL database, or text-files such as files storing the items of data as comma-separated values or in an XML structure.

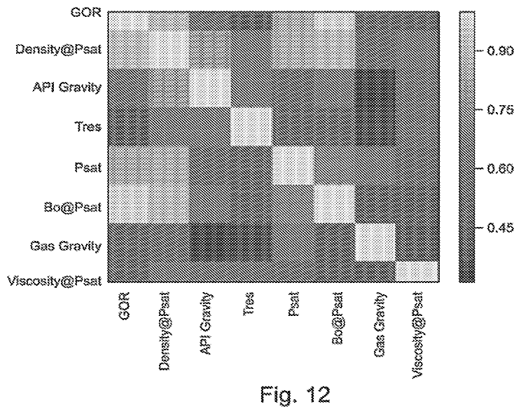

The method 100 further comprises the step of selecting in step 104 items of the PVT data from the incomplete set of PVT data by the reader module 302. The selecting 104 of the items of PVT data is done using exploratory data analysis (EDA). The exploratory data analysis is a method used for automatically analyzing items of data and grouping these items of data into sets of data. This grouping of the data is based on observed patterns within the items of PVT data. These observed patterns in the items of data indicate a relationship between the items of data. The selecting 104 is done based on, for example, items of the PVT data having composition properties or black oil properties that are physically related (see also description of FIGS. 12, 13, 14, 15, 16A and 16B below). The method 100 comprises the step of processing 105 the selected items of the PVT data by a correlating module 303 to identify a plurality of correlations in the selected items of the PVT data based on one or more of the fluid properties of the hydrocarbon fluid samples (see also FIG. 7 showing the workflow diagram of the machine learning module).

The method 100 further comprises the step of data clustering 106, using of at least one of a plurality of clustering schemes, the selected items of the PVT data into a plurality of clusters by a clustering module 304. The data clustering is done using, for example, a K-Means clustering scheme. A cluster of PVT data comprises items of data having similar properties or features. The K-Means clustering scheme is an iterative algorithm that partitions the items of data into distinct and non-overlapping clusters in which each item of data belongs to only a single cluster. The K-Means clustering scheme further comprises determining an arithmetic mean of the items of data in the clusters cluster. This arithmetic mean is called “centroid of the cluster”. The iterations of the K-Means clustering scheme are performed until a sum of squared distances of each of the items of data in the cluster is at a minimum. Other clustering schemes such as density-based spatial clustering of applications with noise, also referred to as “DBSCAN” or hierarchical clustering can also be used. The step of data clustering 106 is used to improve an overall performance of a prediction in the method 100.

The method 100 comprises the step of performing 107 machine learning by a machine learning module 305 on ones of the plurality of clusters to predict missing items of data for the black oil properties and compositional properties in the incomplete set of PVT data and thus to obtain a complete set of PVT data.

The incomplete set of the PVT data for hydrocarbon fluid samples comprises black oil properties and compositional properties. The black oil properties comprise at least one of reservoir temperature, solution gas-oil ratio, oil API gravity, gas gravity, dead oil viscosity, saturation pressure, saturated bubble point oil formation factor at saturation pressure, fluid density at reservoir conditions, fluid compressibility at reservoir conditions, viscosity at reservoir conditions, fluid density at reservoir conditions, and wherein the compositional properties comprise at least one of mole fractions of the components, in particular N2, H2S, CO2, C1, C2, C3, C4, C5, C7, C8, and pseudo-components, in particular C7+, C12+, C20+ and C36+ or any other pseudo-component as well as the molecular weight of the pseudo-components.

The data clustering step 106 of method 100 is applied prior to the step of performing 107 machine learning by a machine learning module 305. The data clustering step 106 is used to categorize the PVT data into families based on their collective behavior of the different features of the PVT data and, hence, improve the quality of the PVT samples properties prediction using machine learning.

The method 100 further comprises the step of performing 107 machine learning by the machine learning module 305. The method 100 further comprises the step of predicting 108 fluid properties for incomplete sets of PVT data. The incomplete sets of the PVT data comprise, for example, only a reduced number of items of data on the compositional properties or the pseudo-components (see above) of the hydrocarbons. These incomplete sets of PVT data are therefore missing some items of the data, for example on the properties or the pseudo-components of the hydrocarbons. The machine learning module 305 is used to predict these missing properties in the incomplete sets of PVT data. This predicting of the missing properties is used, for example, when a new PVT sample is acquired and when this newly acquired new PVT sample does not have a complete set of data and is missing items of data relating to the compositional properties or the pseudo-components. The method 100 comprises the step of plotting 108 the machine learning predictions by the machine learning module 305. The method 100 further comprises the step of comparing 109 the identified plurality of correlation results with the machine learning predictions.

The method 100 according to this first aspect uses algorithms to predict in series, starting from the incomplete set of PVT data (black oil/and compositional) to obtain a complete set of PVT data through method 100 that predicts and uses highest correlating properties first, wherein the highest correlating properties are the properties of the PVT data (black oil/and compositional) having the highest degree of correlation.

The method 100 further comprises that the step of the data clustering 106 further comprises the step of completing fluid composition including C12+, C20+ and C36+ mole fraction and molecular weight and/or completing black oil properties including, in the following order: solution gas oil ratio (GOR), BO@Psat, and saturation pressure (Psat). Thereby, it is possible to learn from existing data and “automatically” complete missing PVT data in every PVT sample using a step-by-step process. The order through which PVT data is completed is set out in the following order and relies on benefiting from the existing PVT data to predict missing PVT data starting from the highest correlating PVT data to the lowest correlating PVT data. The order through which the PVT data is completed allows a minimization of minimize propagation of error from prediction of PVT data in an earlier step of the process to a later step in the process. The following terminology is used:

| | |
|---|---|
| Propᵢₙ | Input properties |
| Propₒᵤₜ | Output properties |
| MLₑᵣᵣₒᵣ_{_{—}}ₜᵣₐᵢₙ | Calculated error resulting from the Training phase of |
| | a given ML method |
| MLₑᵣᵣₒᵣ_{_{—}}ₜₑₛₜ | Calculated error resulting from the Testing phase of |
| | a given ML method |
| Nₚᵢ | Number of samples with the complete set of Propᵢₙ |
| Nₚᵢ_{_{—}}ₜᵣₐᵢₙ | Number of samples with the complete set of Propᵢₙ to be |
| | used in the Training phase of the ML |
| Nₚᵢ_{_{—}}ₜₑₛₜ | Number of samples with the complete set of Propᵢₙ to be |
| | used in the Testing phase of the ML |
| | [symbol] = Nₚᵢ − Nₚᵢ_{_{—}}ₜᵣₐᵢₙ |
| Nₚₒ | Number of samples for which Propₒᵤₜ to be predicted |
| N_{c}_{_{—}}ₜₒₜₐₗ | Total number of samples with compositional information |
| [symbol] | Number of samples with complete C7+ |
| [symbol] | Number of samples with complete C7+ and C12+ |
| [symbol] | Number of samples with complete C7+, C12+ and C20+ |
| N_{c}_{_{—}}_{complete} | Number of samples with complete C7+, C12+, C20+ |
| | and C36+ |
| [symbol] | Number of samples with incomplete C12+ |
| [symbol] | Number of samples with incomplete C20+ |
| N_{c}_{_{complete}} | Number of samples with incomplete C36+ |
| N_{c}_{_{incomplete}} | Number of samples with incomplete compositional data |
| | N_{c}_{_{incomplete}} = N_{c}_{_{—}}ₜₒₜₐₗ − N_{c}_{_{—}}_{complete} |

The output properties Propₒᵤₜ are predicted using the most optimal machine learning method. In particular, as an Input: N_pi samples, N_po samples and Propₒᵤₜ are used to predict for an output: Complete Propₒᵤₜ for N_po samples.

The flowing algorithm is used to predict any missing property Propₒᵤₜ:

ML_{error_min}=big_number

For every ML method
Train to predict Propₒᵤₜ using N_(pi_train) samples
Test using N_(pi_test) samples to test ML's capability to predict Propₒᵤₜ
Calculate ML_{error_test}: if ML_{error_test}<ML_{error_min} [symbol] ML_best=ML Predict Propₒᵤₜ (N_po) using ML_best

In one aspect of the present invention, the PVT data can be completed without the data clustering step 106, however this is not limiting the present invention. To complete composition data without the data clustering step 106, as an input all samples with compositional data can be used, in particular, mole fractions of N2, H2S, CO2, C1, C2, C3, C4, C5, C6 and C7+ as well as C7+MW. The output are samples with completed C12+, C20+ and C36+ mole fraction and molecular weight (for the specific cluster).

The flowing algorithm is used to complete composition data:

ML_optimal(Mole Fraction C12+)
Input: samples with complete C7+ and C12+ Mole Fractions and MW
Output: Completed C12+ Mole Fraction for all samples ML_optimal (MW C12+)
Input: samples with complete C7+ and C12+ Mole Fractions and MW
Output: Completed C12+MW for all samples ML_optimal (Mole Fraction C20+)
Input: samples with complete C7+, C12+ and C20+ Mole Fractions and MW
Output: Completed C20+ Mole Fraction for all samples ML_optimal (MW C20+)
Input: samples with complete C7+, C12+ and C20+ Mole Fractions and MW
Output: Completed C20+MW for all samples ML_optimal (Mole Fraction C36+)
Input: samples with complete C7+, C12+, C20+ and C36+ Mole Fractions and MW
Output: Completed C36+ Mole Fraction for all samples ML_optimal (MW C36+)
Input: samples with complete C7+, C12+, C20+ and C36+ Mole Fractions and MW
Output: Completed C36+MW for all samples

To complete black oil data, as an input already completed C12+, C20+ and C36+ mole fractions and MW can be used. The output are samples with completed black oil properties.

The flowing algorithm is used to complete black oil data:

ML_optimal(GOR)
Input: All samples (Compositional); some missing GOR.
At Output: All samples have GOR

ML_optimal(BO_Psat)
Input: All samples (Compositional and GOR); some missing BO_Psat
At Output: All samples have GOR and BO_Psat

ML_optimal(Psat)
Input: All samples (Compositional, GOR and BO_Psat); some missing Psat
At Output: All samples have GOR, BO_Psat and Psat

In another aspect of the present invention, the PVT data can be completed with the data clustering step 106, however this is not limiting the present invention. To complete composition data with the data from the data clustering step 106, as an input all samples with compositional data can be used, in particular, mole fractions of N2, H2S, CO2, C1, C2, C3, C4, C5, C6 and C7+ as well as C7+MW. As an output, every sample will be associated with one of the clusters identified in the data clustering step 106.

A data completion algorithm is run for a plurality of clustering schemes to complete all missing compositional and black oil data in the clusters cluster. This data completion algorithm is also referred to as “chain algorithm”. The chain algorithm comprises the steps completing the compositional data for the cluster and subsequently completing the black oil data for the clusters cluster. The chain algorithm further comprises the step of calculating a cumulative root mean square error (RMSE), also referred to as “cumulative error”, for the clusters and then selecting the clustering scheme having the smallest cumulative error. The following is a description of the steps of the chain algorithm:

For every Clustering Scheme (CM)
Cluster_Data
For every cluster
   Complete_Composition_Data of samples belonging to that cluster
   Complete_BO_Properties of samples belonging to that cluster
   Calculate cumulative ML_error_min (from all clusters and all properties)
   Select the clustering scheme that results on the lowest cumulative ML_error_min

FIG. 1C illustrates the chain algorithm. The chain algorithm is used to learn from the items of data to complete missing properties of the PVT sample. In FIG. 1C, CMₖ represents the k^{th} clustering scheme assuming there are K different clustering schemes and a total number of C clusters. In step 700 a value fork is set to k=1. Variable k is a counter used in the chain algorithm. Variable k is an integer. A value of the variable ranges from k=1 to k=K. The clustering is then performed using CMₖ in step 701. For every cluster c the compositional properties are completed using the chain algorithm in step 702, where c indicates a number identifying a current cluster. The black oil properties are then determined in step 703. These black oil properties comprise GOR, Bo@Psat, Psat, density @ Psat, and API gravity (in this order). In step 704 the value of c is incremented by 1. In step 705 a check is conducted to see if the black oil properties of the clusters have been calculated by verifying if the conduced to verify if the number identifying the current cluster c is equal to the total number of clusters C. If c is not equal to C, steps 702 to 704 are repeated. If c=C, the chain algorithm continues with step 706. In step 706 the cumulative error over the number of clusters C is calculated. In step 707 the counter k is incremented by 1. In step 708 a check is conducted to verify if the cumulative error is calculated for all of the clustering schemes by checking whether the counter k is equal to the number of K. If k is not equal to K, steps 701 to 708 are reiterated. If k=K the clustering method with the lowest cumulative error is selected in step 709.

In the chain algorithm, items of data generated in one step are used by a successive step. For instance, the mole fraction of C12+ is predicted using compositional properties up to C7+. Several machine learning (ML) algorithms are evaluated and the ML algorithm with the lowest cumulative error is selected. These ML algorithms are, for example, open source ML algorithms such as Scikit-learn. Scikit-learn is an open source machine learning library that supports supervised and unsupervised learning and comprises a plurality of modules. The ML algorithms are, for example, linear regression algorithms, support vector regression algorithms, K neighbors regression algorithms, regression tree algorithms, extra tree algorithms, random forest algorithms, gradient boosting algorithms, multi-layer perceptron algorithms, bagging algorithms, or AdaBoost algorithms. The selected ML algorithm is then trained on all the complete data to predict the missing values pertaining to the mole fraction of C12+. At this stage, all samples have a complete mole fraction of C12+. This property is then used as input for the later steps. The molecular weight of C12+ using compositional properties up to C7+ along with the mole fraction of C12+ is then predicted. The following table shows the inputs and output of the chain algorithm.

| Step in | | |
|---|---|---|
| Chain | | |
| Algorithm | Inputs | Output |
| 1 | N₂, H₂S, CO₂, C1, C2, C3, C4, C5, C6, mole fraction of C7+, | mole fraction of |
| | molecular weight of C7+ | C12+ |
| 2 | N₂, H₂S, CO₂, C1, C2, C3, C4, C5, C6, mole fraction of C7+, | molecular weight |
| | molecular weight of C7+, mole fraction of C12+ | of C12+ |
| 3 | N₂, H₂S, CO₂, C1, C2, C3, C4, C5, C6, mole fraction of C7+, | mole fraction of |
| | molecular weight of C7+, mole fraction of C12+, molecular | C20+ |
| | weight of C12+ | |
| 4 | N₂, H₂S, CO₂, C1, C2, C3, C4, C5, C6, mole fraction of C7+, | molecular weight |
| | molecular weight of C7+, mole fraction of C12+, molecular | of C20+ |
| | weight of C12+, mole fraction of C20+ | |
| 5 | N₂, H₂S, CO₂, C1, C2, C3, C4, C5, C6, mole fraction of C7+, | mole fraction of |
| | molecular weight of C7+, mole fraction of C12+, molecular | C36+ |
| | weight of C12+, mole fraction of C20+, molecular weight of | |
| | C20+ | |
| 6 | N₂, H₂S, CO₂, C1, C2, C3, C4, C5, C6, mole fraction of C7+, | molecular weight |
| | molecular weight of C7+, mole fraction of C12+, molecular | of C36+ |
| | weight of C12+, mole fraction of C20+, molecular weight of | |
| | C20+, mole fraction of C36+ | |
| 7 | N₂, H₂S, CO₂, C1, C2, C3, C4, C5, C6, mole fraction of C7+, | GOR |
| | molecular weight of C7+, mole fraction of C12+, molecular | |
| | weight of C12+, mole fraction of C20+, molecular weight of | |
| | C20+, mole fraction of C36+, molecular weight of C36+ | |
| 8 | N₂, H₂S, CO₂, C1, C2, C3, C4, C5, C6, mole fraction of C7+, | Bo@Psat |
| | molecular weight of C7+, mole fraction of C12+, molecular | |
| | weight of C12+, mole fraction of C20+, molecular weight of | |
| | C20+, mole fraction of C36+, molecular weight of C36+, | |
| | GOR | |
| 9 | N₂, H₂S, CO₂, C1, C2, C3, C4, C5, C6, mole fraction of C7+, | Psat |
| | molecular weight of C7+, mole fraction of C12+, molecular | |
| | weight of C12+, mole fraction of C20+, molecular weight of | |
| | C20+, mole fraction of C36+, molecular weight of C36+, | |
| | GOR, Bo@Psat | |
| 10 | N₂, H₂S, CO₂, C1, C2, C3, C4, C5, C6, mole fraction of C7+, | Denisty@Psat |
| | molecular weight of C7+, mole fraction of C12+, molecular | |
| | weight of C12+, mole fraction of C20+, molecular weight of | |
| | C20+, mole fraction of C36+, molecular weight of C36+, | |
| | GOR, Bo@Psat, Psat | |
| 11 | N₂, H₂S, CO₂, C1, C2, C3, C4, C5, C6, mole fraction of C7+, | API Gravity |
| | molecular weight of C7+, mole fraction of C12+, molecular | |
| | weight of C12+, mole fraction of C20+, molecular weight of | |
| | C20+, mole fraction of C36+, molecular weight of C36+, | |
| | GOR, Bo@Psat, Psat, Denisty@Psat | |

FIG. 1D is an overview of the correlation of the composition properties over the mole fraction C7+. The composition properties are predicted in the order of the mole fractions and the molecular weights are forecast in an ascending order. The predicting starts by completing composition features sequentially using the following order: mole fraction of C12+, molecular weight of C12+, mole fraction of C20+, molecular weight of C20+, mole fraction of C36+, and molecular weight of C36+. After completing the composition data, the black oil properties are forecast. It can be seen from FIG. 1C that GOR is the feature having the highest degree of correlation to the remaining black oil properties. The degree of correlation of the features in descending order is Bo@Psat, Psat, density @ Psat, and API gravity.

FIG. 1E is an overview of the correlation of the composition properties over the molecular weight C7+.

FIG. 1F shows a flow diagram describing the steps of the ML algorithm. The ML algorithm is applied to the outputs in every cluster. In the flow chart, M denotes the total number of machine learning models used. The ML algorithm is used to determine the missing properties Propₒᵤₜ for the sample Nₚₒ. Samples having the complete set of PVT data are denoted by Nₚᵢ. The samples having the complete PVT data Nₚᵢ are used for the training of the ML algorithm. The missing properties Propₒᵤₜ are then determined (see step 812 below) using the trained ML algorithm. The samples having complete PVT data Nₚᵢ are divided into training data and test data. For example, 80% of the items of data from the Nₚᵢ are used as the training data and, for example, 20% of the items of data from the Nₚᵢ are used as test data. The items of data from the Nₚᵢ can also be divided in other ratios.

In step 800, the items of data Nₚᵢ are split into items of data used as training data N_{pi,train} and items of data used as test data N_{pi,test}. The training data is used for the training of hyperparameters of the ML algorithms. The hyperparameter is a parameter whose value is used to control a learning process of the ML algorithm. These hyperparameters are also referred to as “tuning parameters” for the ML algorithm. The test data is not used for the testing but kept until the training is finished. The test data is then used for a final evaluation of the ML algorithms. The test data is therefore used to quantify how the ML algorithms will perform on unclassified items of data. The objective of this testing is to select the ML algorithm with the least error on the unclassified items of data.

The input properties for the ML algorithm are then selected using the EDA in step 801. The input properties are selected by the EDA based on a degree of correlation between these input properties. A value for a minimal error is set as a big number, for example greater than 1,000. in step 802 and a value for m is set to m=1. Variable m is used as a counter and is, for example, an integer. After the selecting of the input properties, the hyperparameter of the ML algorithm are tuned in step 803. The tuning in step 803 of these hyperparameters is done automatically by the ML algorithm. In step 804 every ML algorithm is trained using the training data.

In step 805 the ML algorithm is tested using the test data N_{pi,test} For example, a grid search approach with five folds of cross validation is used to select the parameters for every ML algorithm. In this validation, the training data is divided into five equal size subsets where each subset serves as validation data exactly once. For every possible combination of the hyperparameters, the ML algorithm is trained on four subsets of the training data and validated on the remaining one subset of the training data. This will result in five error values—one for every fold or subset—for every combination of the hyperparameters. An average of these five error values is then calculated in step 806. This average describes an error value for every set of hyperparameters. The hyperparameters with the least average error from the five folds or subset are then selected as parameters for the selected ML algorithm.

The ML algorithm is then evaluated on the unclassified items of the test data in step 807. Using the error values for the ML algorithm calculated using, for example, R² and root mean square error (RMSE) methods, the ML algorithm is then evaluated. R² describes a proportion of a variance in a response variable that can be explained by a predictor variable. R² is used to evaluate numerical predictions an amount of variance explained by ML algorithm. The R² is calculated using the following equation, where n is the number of items of data in the data set, yᵢ is the value of the item of data i:

$${R}^{2} = 1 - \frac{{\sum }_{i = 1}^{n} {( {y}_{i} - {y}_{i}^{′} )}^{2}}{{\sum }_{i = 1}^{n} {( {y}_{i} - μ )}^{2}}$$

The value for μ is calculated using the following equation:

$$μ = \frac{1}{n} {\sum }_{i = 1}^{n} {y}_{i}$$

The RMSE is a metric for quantifying an average distance between predicted items of data from the ML algorithm and actual items of data in the data set.

$$R MSE = \sqrt{\frac{1}{n} {\sum }_{i = 1}^{n} {( {y}_{i} - {y}_{i}^{′} )}^{2}}$$

Using the R² or the RMSE, the ML algorithms are evaluated before the selecting in step 808 of the ML algorithm. This evaluation may also be done using, for example, linear regression, support vector regression, regression tree, Random Forest, gradient boosting, AdaBoost, Bagging, neural networks, and ensemble model. The selecting in step 808 of the ML algorithms is, for example, done by selecting the ML algorithm with a smallest value for the RMSE when applied to the test data. This selecting is performed for every output in every cluster. After the selecting of the ML algorithm, the counter m is incremented by 1 in step 809. In step 810 is conducted to verify if the counter m is equal to M, wherein M is a total number of ML algorithms used in this analysis. If m is not equal to M in step 810, steps 803 to 810 are reiterated. If m=Min step 810, the ML algorithm is retrained with all samples from the Nₚᵢ in step 811. The missing properties Propₒᵤₜ are then determined in step 812.

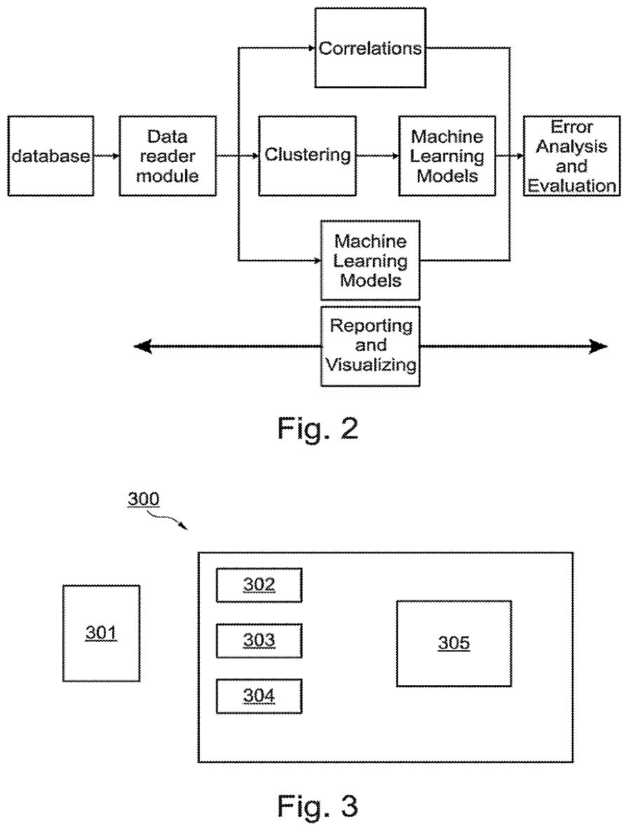

FIG. 2 is a schematic illustration of the overall view of the workflow of the computer-implemented method for predicting hydrocarbon fluid properties using machine-learning-based models in accordance with the first aspect of the present invention.

The PVT data set is parsed into a standard format and passed to later components in the data reader module 305. The step of data clustering 106 divides the PVT data from the PVT data set into groups such that samples in one cluster are likely to be similar. This unravels hidden insights and facilitates learning and improve performance Empirical correlations available in the literature can be applied to provide reference results (in terms of predictive capability) to the final machine learning results. The step of machine learning can be performed on the whole PVT data set at once. More importantly, machine learning can be performed on every one of the clusters to predict missing PVT properties and prepare for predicting PVT properties for incomplete PVT data. As can be seen in FIG. 2, analysis is performed to compare machine learning predictions with empirical correlations. Further, logging and reporting (e.g. through production of files in Excel, PDF and JPEG) is taking place at every step.

FIG. 3 is a schematic illustration of a system 300 for predicting hydrocarbon fluid properties using machine-learning-based models in accordance with a second aspect of the present invention. FIG. 3 shows the system 300 for predicting hydrocarbon fluid properties using machine-learning-based models, wherein the system 300 comprises a pressure-volume-temperature (PVT) data base 301, a reader module 302, a correlating module 303, a clustering module 304, and a machine learning module 305. The pressure-volume-temperature (PVT) data base 301 provides the incomplete set of pressure-volume-temperature (PVT) data for the hydrocarbon fluid samples. The reader module 302 reads in the incomplete set of the PVT data. The reader module 305 is configured to transform the incomplete set of PVT data into a unified data structure, and the reader module 30 is configured to select items of the PVT data from the incomplete set of PVT data. The correlating module 303 processes the selected items of the PVT data to identify a plurality of correlations in the selected items of the PVT data based on one or more of the fluid properties of the hydrocarbon fluid samples. The clustering module 304 clusters, using at least one of a plurality of the above clustering schemes, the selected items of the PVT data into a plurality of clusters. The machine learning module 305 performs machine learning on ones of the plurality of clusters to predict missing fluid properties in the incomplete set of PVT data and thus to obtain a complete set of PVT data.

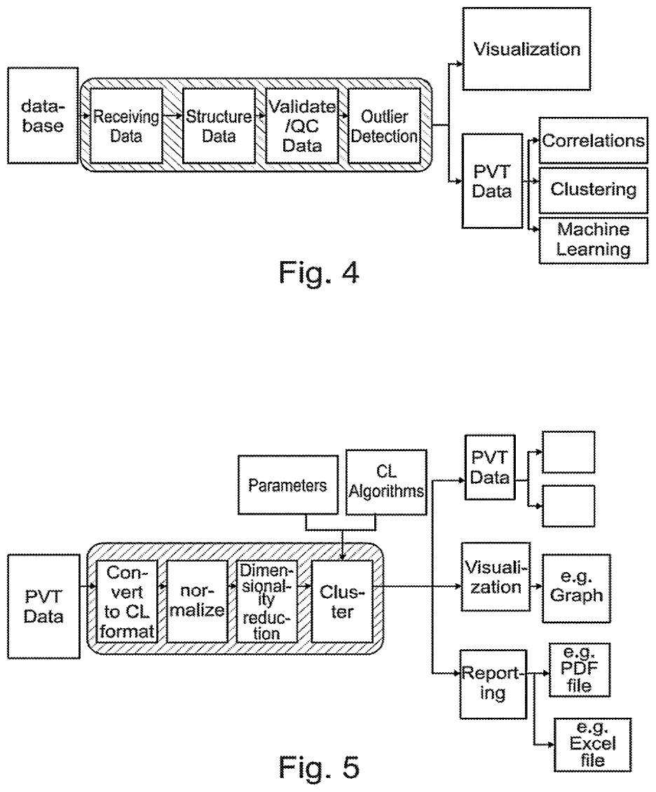

FIG. 4 shows a workflow of the data reader module 302. PVT Data is provided from the PVT data base 301 (e.g., in excel format). The desired PVT properties are parsed and extracted. The PVT data is then transformed into a unified data structure to undergo further processing. A quality check is performed to account for out of range properties (e.g. negative values), duplicate samples, etc., wherein out of range values (e.g., API_gravity 0→100) are considered outliers. The statistics are visualized and reported. The validated PVT data will proceed to further components such as clustering, machine learning, correlations, etc.

FIG. 5 shows a workflow of the clustering module 303. The required PVT data structure is received from the data reader module 302. The received data is converted into a custom format on which the step of data clustering 106 can operate. This custom format is substantially a 2D matrix in which each row represents a sample, and properties of the sample are represented by columns. PVT properties with different ranges can screw clustering models since properties having high ranges are considered of high importance. Therefore, normalizing data to have zero mean and unit variance is necessary. In high dimensional spaces, the step of data clustering 106 may not work well, hence, dimensionality reduction might be useful. The step of data clustering 106 will be performed using several clustering schemes available in the literature, as described above. The clustering results and comparisons (e.g., properties before versus properties after clustering) are visualized. Figures are reported in e.g., using PDF documents as an output, whereas resulting tables are reported in e.g. an excel that can be later imported in a visualization program such as Spotfire. The PVT data structure is updated with its cluster ID and passed to other components such as the machine learning module 305.

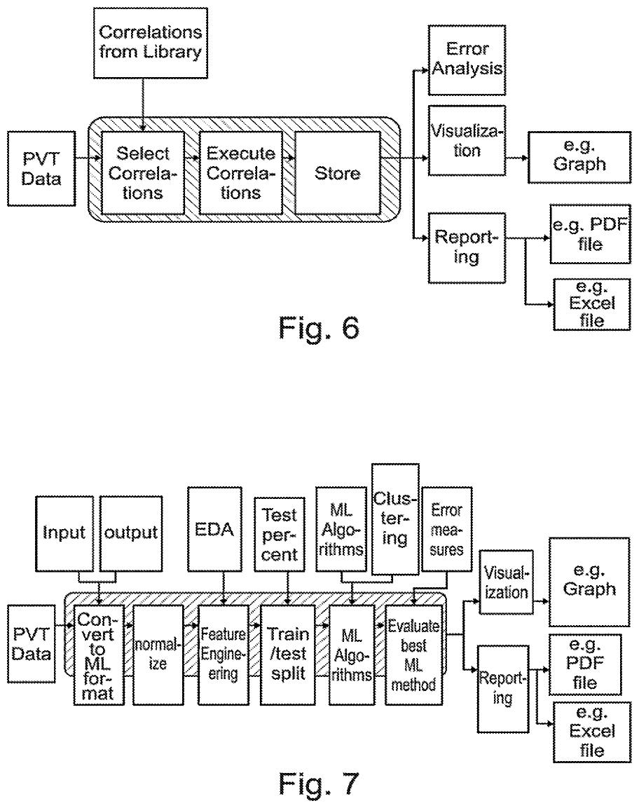

FIG. 6 shows a workflow diagram of the correlating module 303. The required PVT data structure is received from the data reader module 302. A plurality of correlations found in the literature are processed for particular properties (Psat, Bo@Psat and Dead oil viscosity).

The following correlations are processed in the present invention:
Psat
Standing (Standing 1947)
Al Marhoun (Al-Marhoun 1988)
Vazquez and Beggs (Vazquez and Beggs 1980)
Kartoatmodjo and Schmidt (Kartoatmodjo and Schmidt 1994)
Dokla and Osman (Dokla and Osman 1992)
Petrosky Jr. Farshad (Petrosky Jr and Farshad 1993)
Al Shammasi (Al-Shammasi 2001)
Dindoruk & Christman (Dindoruk and Christman 2001)
Khamehchi et al. (Khamehchi, Rashidi et al. 2009)
Arabloo et al. (Arabloo, Amooie et al. 2014)
Jarrahian et al. (Jarrahian, Moghadasi et al. 2015)
Bo@Psat
Standing (Standing 1947)
Al Marhoun (Al-Marhoun 1988)
Vazquez and Beggs (Vazquez and Beggs 1980)
Kartoatmodjo and Schmidt (Kartoatmodjo and Schmidt 1994)
Dokla and Osman (Dokla and Osman 1992)
Petrosky Jr.F Arshad (Petrosky Jr and Farshad 1993)
Omar and Todd (Omar and Todd 1993)
Al Mehaideb (Almehaideb 1997)
Al Shammasi (Al-Shammasi 2001)
Dindoruk and Christman (Dindoruk and Christman 2001)
Ikiensikimama and Ajienka (Ikiensikimama and Ajienka 2012)
Arabloo et al. (Arabloo, Amooie et al. 2014)
Dead oil viscosity
Beggs and Robinson (Beggs and Robinson 1975)
Glaso (Glaso 1980)
Dindoruk and Christman (Dindoruk and Christman 2001)
Naseri et al. (Naseri, Nikazar et al. 2005)
Kartoatmodjo and Schmidt (Kartoatmodjo and Schmidt 1994)
Petrosky Jr. Farshad (Petrosky Jr and Farshad 1993)
Labedi (Labedi 1992)

As can be seen in FIG. 6, the selected correlations are processed and the resulting values from each of the selected correlations are for example returned in a vector format. The resulting vectors are stored in a table format for every PVT data and for every property. At a later stage, the obtained correlation values will be compared with the machine learning results. Errors are calculated using the resulting values from the empirical correlations and the real values. The errors are visualized graphically. The figures are reported e.g., in a PDF output file, whereas resulting tables are reported in e.g., an excel format, that can be later imported to visualization tools (e.g. in Spotfire).

FIG. 7 shows a workflow of the machine learning module 305. The required PVT data structure is received from data reader module 302. The received PVT data is converted into a custom format that the machine learning models can operate on, e.g., the afore-mentioned 2D matrix in which each row represents a sample, and properties are represented by columns. The properties with different ranges mess up the machine learning models, as mentioned above and therefore, normalizing PVT data to have zero mean and unit variance is necessary. For example, Exploratory Data Analysis (EDA) can be utilized to conclude PVT properties correlations and which the PVT properties affect a particular output the most. To ensure model generalization, the PVT data is divided into two parts, training data and evaluation data. Several machine learning methods are trained. If clustering preceded machine learning, training is applied on every individual cluster. The best machine learning model is chosen based on minimum error (e.g., RMSE) on the test (hidden) data. The machine learning predictions are plotted and compared with correlations. Further, FIGS. can be reported e.g. in a PDF, whereas resulting tables can be reported e.g. in an excel that can be later imported, e.g., in Spotfire.

The following list of machine learning can be processed with the present invention, however, the present invention is not limited thereby.

### LR=Linear Regression;

### SVR=Support Vector Regression;

### KN=K Neighbors Regression;

### RT=Regression Tree;

### ET=Extra Tree;

### RF=Random Forest;

### GB=Gradient Boosting;

### MLP=Multi-layer Perceptron;

### Bagging;

### Adaboost; and

### Ensemble.

For achieving the expected result in the present invention, the following steps are proceeded. In the first step the code design is ingested, whereas the PVT data base 301 is read. The PVT data base 301 is structured for example as multiple excel files as follows: PVT Project: containing information about the project of which the fluid sample was taken such as the project's name, the year, the laboratory in which the experiment was done, the well from which the sample was taken, etc.; PVT Black Oil Properties: containing the black oil properties for each fluid sample such as the oil gravity API, reservoir temperature, pressure, bubble point pressure, oil formation volume factor at the bubble point pressure, etc.; Well Coordinates: containing entries of all the wells with the X and Y coordinates of each; and Compositions: containing the molecular composition for each fluid sample for the three sample types which are the “Reservoir Field”, “Evolved Gas”, and “Stock-Tank Oil”. The molecular composition of the heavy components is lumped.

In order to have the complete PVT data for each of the fluid samples, the PVT data from the different excel files are merged and linked to each other using the project ID which is unique for each project. Therefore, the PVT project and PVT Black oil properties are merged using the project ID as unique key for the sample. To add the X and Y coordinates to each fluid sample, each sample is linked to the Well Coordinates excel file with its well name. Then, the coordinates of that well are associated with the sample. The composition of each sample is also linked with previous data using the project ID. The mole fractions of C7+, C12+, C20+, and C36+ are calculated as follows: Mole fraction of C7+=mole fraction of M-C-05+ . . . + mole fraction of C36; Mole fraction of C12+=mole fraction of C12+ . . . + mole fraction of C36; Mole fraction of C20+=mole fraction of C20+ . . . + mole fraction of C36; Mole fraction of C36+=mole fraction of C36. However, heavy components are lumped, and the heavy component (Plus Fraction) is indicated in the PVT project excel file. Therefore, when the heavy component is reached, all other mole fractions should be zero. The molecular weights of C7+, C12+, C20+, and C36+ are also calculated by performing several intermediate calculations and using the previously calculated mole fractions.

In the second step data a screening and quality check (QC) is applied. The screening and quality check mechanism is applied to better understand the PVT data and to ensure that the PVT data does not contain any anomalies. The PVT data comprises data from the hydrocarbon fluid samples that are complete as well as hydrocarbon fluid samples that are missing values of the data, e.g., values being set to zero in the sample. The PVT data is therefore grouped into two categories. The first category in the PVT data are values for the hydrocarbon fluid samples that exist but are absent from the samples, such as Psat or GOR. The values of this first category can occur when items of data are “lumped”. These values are, for example, a C20+ mole fraction for a hydrocarbon liquid sample. This value must always be >0. The value might, however, be shown as =0 because of grouping that has been applied to the sample. The value might also be shown as =0 because the compositional analysis has only been performed up to C7+. The molecular weight for these absent heavy fractions is set to zero in the dataset. The second category of PVT data comprises values for the hydrocarbon fluid samples that are missing because the data does do not physically exist for these samples. For example, Bo@Psat does not exist for a hydrocarbon vapor sample.

This quality check step is done before the PVT data is used by the clustering schemes and machine learning algorithms. The PVT data identified with anomalies are flagged so that this data with the anomalies are not used in the clustering module 303 and machine learning modules 305. The steps of screening and QC comprises the following: Identifying the anomalies in the input PVT data i.e., unphysical values; Flagging of the PVT data and preparing data structure with proper indicators on complete and missing properties from each of the PVT data. This step is carried out before passing the information to the clustering schemes and machine learning modules. This enables the completion of the information once machine learning models are built.

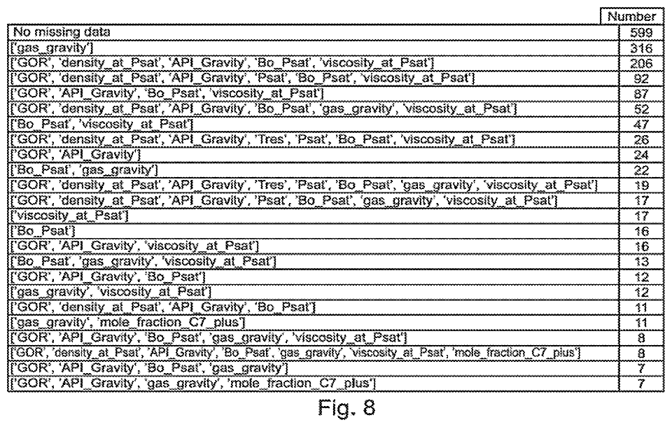

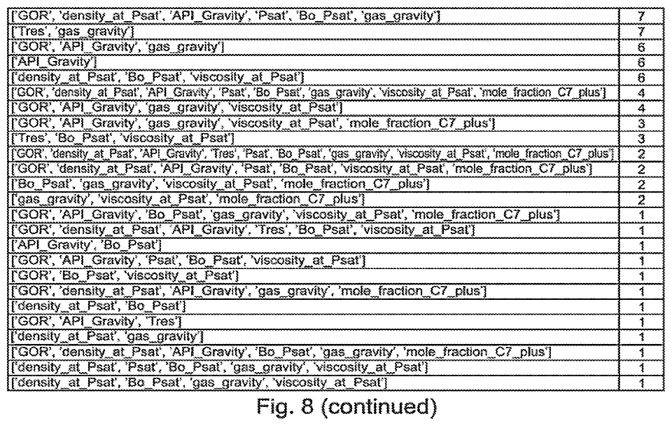

The results of the screening and the QC of the PVT data base 301 are the following: The total number of PVT data is, for example, 1711. With respect to the black oil properties, 593 out of 568 samples have a complete black oil (BO) set of properties with regard to the black oil properties, as can be seen in FIG. 8 (table). The number of the samples will increase or shrink depending how many properties are considered in the machine learning module 305. With respect to the compositional information, the number of the samples with valid composition to be used in machine learning module 305 is 1599. A missing heavy fraction, however, does not mean that the heavy fraction does not exist, moreover a “lumped” sample including the missing heavy fraction is existing.

With respect to the dynamic data ingestion, every step uses the maximum number of samples. For example, when machine learning is used to predict Psat from “Composition” properties only, all the samples that have values for Psat and Composition should be used. These samples may not have Bo or other Black Oil properties. Accordingly, the step during which samples to be tagged complete/incomplete is a “dynamic” tag rather than a “static” tag. For machine learning, Clustering or EDA, this tagging step is performed once “Run” has been clicked. The general rules for samples with Mole Fraction C7+, C12+, C20+ or C36+=Zero are as follows: Mole Fraction of C7+, C12+, C20+ or C36+ are typically >0 for Hydrocarbon Liquid samples; Mole Fraction of C36+ is typically =0 for Hydrocarbon Vapor (gas) samples. Mole Fraction of C20+ is typically =0 for Hydrocarbon Vapor (gas) samples unless we have a gas condensate reservoir; Mole Fraction of C7+ is typically >0 for Hydrocarbon Vapor (gas) samples unless we have dry gas (predominantly methane); Molecular Weight is >>0. However, it should be “meaningless”, when the mole fraction=0. It should not be used in this case. Therefore, mole fraction of C36+ may be 0. It is a valid value (given the above). However, in this case, the molecular weight of C36+ for that specific sample should not be used and is considered undefined. This particular case exists in the case of EDA and Clustering. It does not exist in the case of machine learning. The following observations can be made when looking at the Psat, Bo, and mole fractions of C1, C7+, C12+, C20+ and C36+: Since Bo is an Oil property, it is unlikely to have samples (with defined Bo) with mole fraction of C7+, C12+, C20+ or C36+=0; For samples with defined Bo, the mole fraction of methane is typically <50% or 40%.

In the third step, data analysis is performed to generate statistics about the PVT data. This helps to better understand the distribution of the PVT data to be able to evaluate and interpret the PVT data. The following statistics and property distributions are automatically generated and reported using this framework: Distribution of the data across the fields; Distribution of the data across the reservoirs; Missing Values for each set of properties; Data distribution of the black oil properties, e.g., Saturation Pressure, API Gravity, Reservoir Temperature, Saturated bubble point oil formation volume factor, GOR, Density at saturation pressure, Viscosity at saturation pressure, Data distribution of Compositional Properties (Mole fraction of C7+, Mole fraction of C12+, Mole fraction of C20+, Mole fraction of C36+, Molecular weight of C7+, Molecular weight of C12+, Molecular weight of C20+, Molecular weight of C36+).

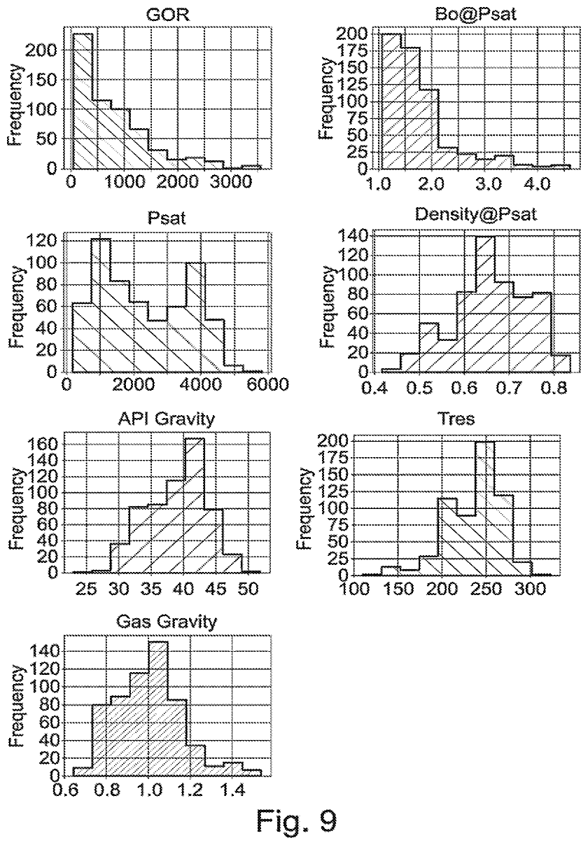

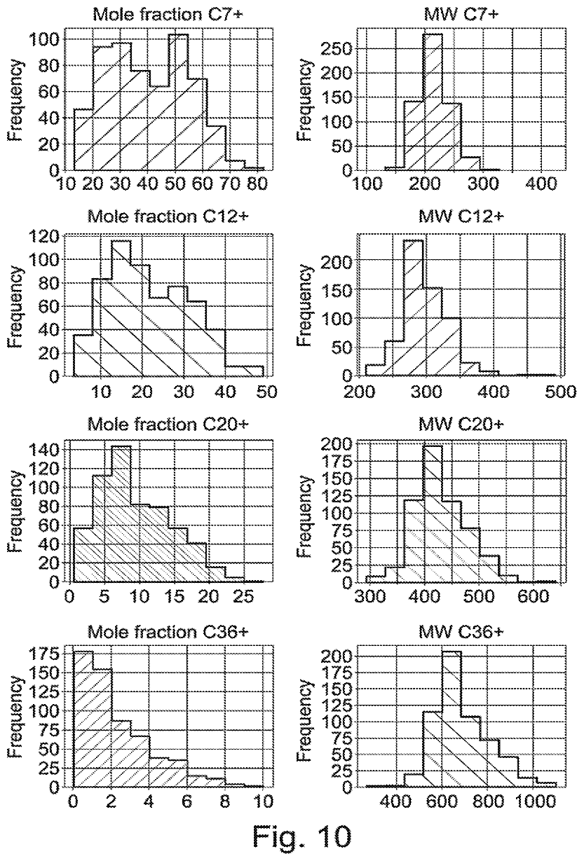

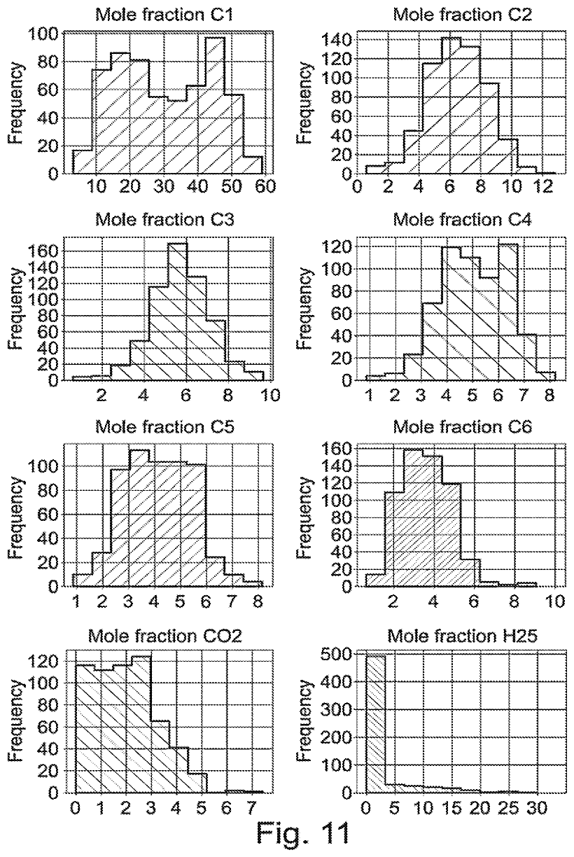

Examples of data statistics are presented in the FIGS. 9, 10 and 11. FIG. 9 is a PVT Data set used in the implementation and validation process, in particular data statistics and black oil properties. FIG. 10 is a PVT Data set used in the implementation and validation process, particular data statistics, compositional properties, and heavy fractions. FIG. 11 is a PVT Data set used in the implementation and validation process, in particular data statistics, compositional properties, light and intermediate components.

With respect to the data prediction, algorithms are used to predict, using machine learning methods, any PVT property (Black oil or compositional) from any set of properties (black oil or compositional). Any of the properties in FLUID PROPERTIES can be predicted as a function of a sub-set of all other properties. The algorithm is generic and allows for adding any other property that can be obtained in a laboratory analysis (e.g. CCE, CVF, DL, MSS) at any pressure.

For validation, the present method 100 runs first machine learning to predict Psat and Bo@Psat from the same parameters used in the literature to predict these two parameters with correlations: temperature, gas oil ratio, stock tank API gravity and gas gravity. For understanding dependencies and eliminating irrelevant features, exploratory data analysis (EDA) is used in the present invention to decrease the number of features used in PVT data prediction. The EDA explores correlation between parameters to, systematically, eliminate parameters with no correlation from the machine learning models. Example results are presented in FIGS. 12, 13, 14, 15, 16A, and 16B.

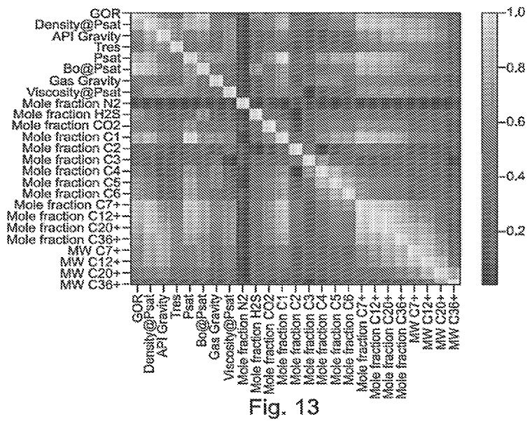

FIGS. 12 and 13 are the results of the EDA applied to the PVT data set used in the implementation and validation process, in particular FIG. 12 for black oil properties and FIG. 13 for black oil and compositional properties. The EDA results shown in FIG. 12 and FIG. 13 indicate a degree of correlation between the black oil properties (see FIG. 12) or the black oil properties and the compositional properties (see FIG. 13). The degree of correlation is indicated by a darkness of a field in FIG. 12 and FIG. 13. A high degree of correlation is indicated by a light shading, a low degree of correlation is indicated by a dark shading of the field. As can be seen from FIG. 12, there is a high degree of correlation between GOR and Bo@Psat.

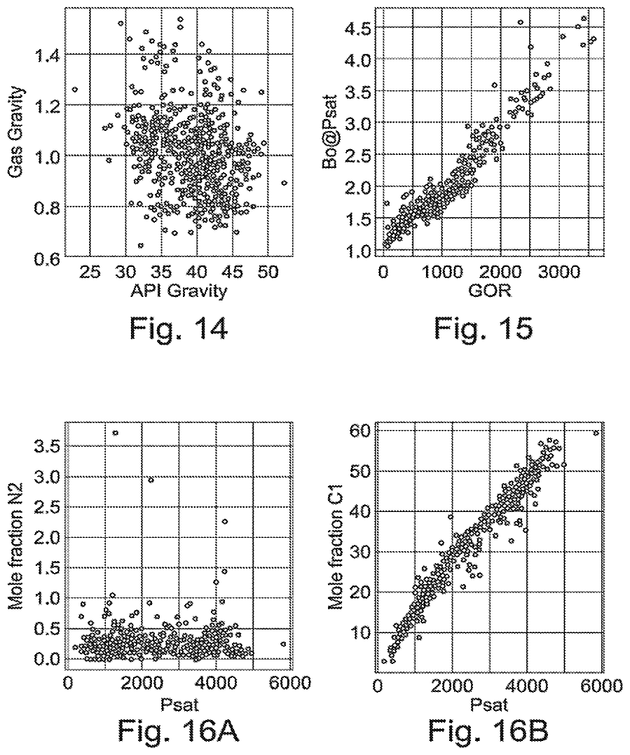

FIGS. 14 and 15 are EDA Results for the black oil properties shown as a correlation cross-plot. As can be seen from FIG. 14, there is a low degree of correlation between the API gravity and the gas gravity. As can be seen from FIG. 15 there is a high degree of correlation between the GOR and the Bo@Psat (as was already indicated in FIG. 12). The properties included in the EDA of FIGS. 14 and 15 are illustrated in FIG. 12.

FIGS. 16A and 16B are the EDA results for the compositional properties shown as a correlation cross-plot. As can be seen from FIG. 16A there is a low degree of correlation between an Psat and the mole fraction N2. As can be seen from FIG. 16B, there is a high degree of correlation between Psat and the mole fraction C1. The properties included in the EDA of FIGS. 16A and 16B are illustrated in FIG. 13.

With respect to improved accuracy prediction using the data clustering step 106, the data clustering step 106 is used to categorize the PVT data into families based on their collective behavior of their different features. The data clustering step 106 is performed for two main reasons: Using machine learning on different clusters instead of the whole PVT data set with the purpose of improving the predictive capability of different methods for different clusters; the EoS models available for different clusters will be compared with each other. In case similarity is found between these EoS models, one representative EoS model is selected per cluster. The EoS models are thermodynamic models used for predicting the fluid properties under an expected range of pressure and a temperature covering a life of a reservoir. Once every sample belongs to a given cluster, the representative EoS model for that specific sample will be adopted for the specific sample.

The clustering 106 of method 100 can be performed using the following options: Clustering using black oil properties only; Clustering with compositional properties only; and Clustering with black oil and compositional properties. In all three cases, the clustering results can be used for machine learning to predict any of the black oil or compositional properties. In the following, the clustering to predict black oil properties is described to find out whether black oil properties can be predicted using compositional properties only without impacting the quality of the prediction.

For black oil clustering, the properties used for clustering are the following. Any other property could be used. Similarly, less properties or any other combination of properties can also be used.

Reservoir Temperature (Tres);

Solution gas oil ratio (GOR);

API Gravity;

Gas Gravity;

Saturation pressure (Psat);

Bo@Psat;

Viscosity @ Psat; and

Density @ Psat.

The number of samples used in this case is 593 samples. These are the samples with all above properties available.

For compositional properties based clustering, clustering is performed using completed compositional properties for which the compositional information is available for the full set of 1599 samples. This leads to a total of 1599 samples used in the clustering. Optimal number of clusters is this case is 5.

For black oil and compositional based clustering, clustering is performed using the following black oil properties:
Reservoir Temperature (Tres);
Solution gas oil ratio (GOR);
API Gravity;
Gas Gravity;
Saturation pressure (Psat);
Bo@Psat;
Viscosity @ Psat; and
Density @ Psat.

Completed compositional properties for which the compositional information is available for the 593 samples. The total number of samples that have all above data is 593.

Further, clusters can be associated with fields and reservoirs. The association between clusters of PVT data identified through the clustering step oil and gas fields/reservoirs with potentially different thermodynamic behavior.

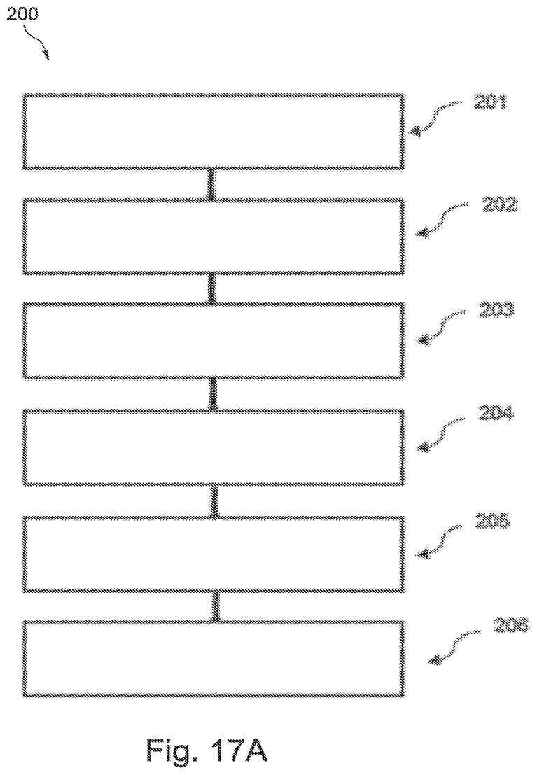

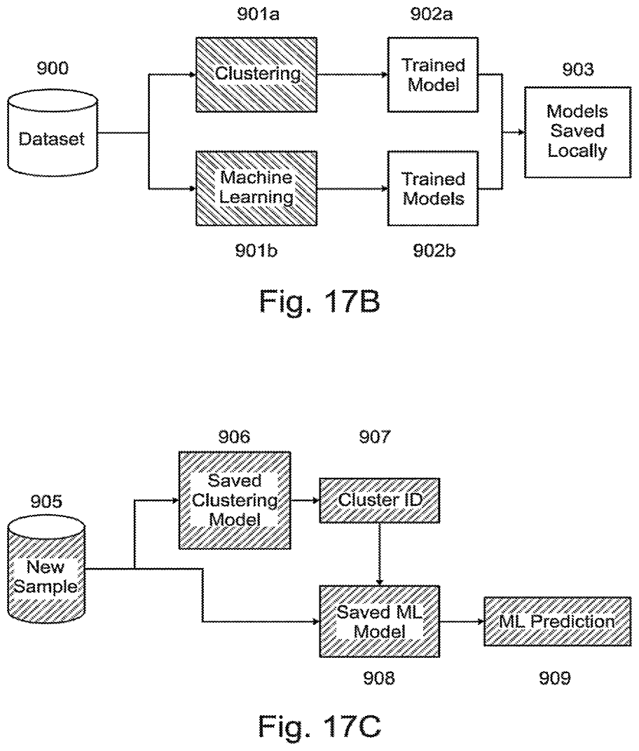

FIG. 17 is a flow diagram of a computer-implemented method 200 for generating equations of state (EoS) for a plurality of hydrocarbon fluids in accordance with a third aspect of the present invention. The computer-implemented method 200 for generating the equations of state (EoS) for a plurality of hydrocarbon fluids comprises the method step of delumping 201 pressure-volume-temperature (PVT) data for hydrocarbon fluid samples from a complete set of PVT data to one of a set of detailed fluid components, or to a common set of components and pseudo-components. The complete set of PVT data can be provided by the computer-implemented method 100 for predicting hydrocarbon fluid properties using machine-learning-based models in accordance with the first aspect of the present invention.

Further, the method 200 comprises the step of lumping 202 the PVT data from the complete set of PVT data into a pre-defined set of components and pre-defined set of pseudo-components to generate a plurality of equation of state (EoS) models. Further, the method 200 comprises generating 203 for the PVT samples on the PVT data set an EoS model using a same set of tuning parameters and thereby generating an EoS fluid model fingerprint for the hydrocarbon fluid samples. The EoS fluid model fingerprint refers to a specific heavy fraction characterization of the PVT data that defines a specific EoS model. The properties of the heavy fractions are then tuned or adjusted to match the laboratory data leading to unique characteristics of that specific fluid and its corresponding EoS model. Further, the method 200 comprises the step of associating 204 properties of the hydrocarbon fluid samples with the generated EoS fluid model fingerprint.

The method 200, as shown in FIG. 17, comprises the step of training 205 machine learning models to predict an EoS fluid model fingerprint for new hydrocarbon fluid samples. These new hydrocarbon fluid samples are samples not having been used for the training of the ML algorithm. The incomplete set of PVT data for hydrocarbon fluid samples comprises black oil properties and compositional properties, wherein the black oil properties comprise at least one of reservoir temperature, solution gas-oil ratio, oil API gravity, gas gravity, dead oil viscosity, saturation pressure, saturated bubble point oil formation factor at saturation pressure, fluid density at reservoir conditions, fluid compressibility at reservoir conditions, viscosity at reservoir conditions, fluid density at reservoir conditions or any other black oil property, and wherein the compositional properties comprise at least one of mole fractions of the components, in particular N2, H2S, CO2, C1, C2, C3, C4, C5, C7, C8, and pseud-components, in particular C7+, C12+, C20+ and C36+ or any other pseudo-component as well as the molecular weight of the pseudo-components.

The step of associating 204 of method 200 further comprises the method step of clustering the selected items of the PVT data into a plurality of clusters for performing machine learning on each one of the plurality of clusters.

The method 200 further comprises the step of comparing 205 the results from clustering of the selected items of the PVT data with the plurality of equations of state (EoS) models and with heatmaps applying EoS models on the selected items of the PVT data.

The clustering 106, 204 of the selected items of the PVT data includes the step of identifying of clusters to which PVT data belong.

For delumping 201 into a common set of detailed components, as an input, the method step starts with SAMPLES that may or may not have the same set of DETAILED COMPOSTION or to a common set of components and pseudo-components. The method 100 according to the first aspect of the present invention is used to complete all SAMPLES into the same set of detailed components or to a common set of components and pseudo-components. The completing of the SAMPLES is done using the chain algorithm.

As a first example, some samples would have the following set of components and pseudo-components: CO2, H2S, N2, C1, C2, C3, iC4, nC4, iC5, nC5, C6, C7+. Mole fraction of C7, C8, . . . , C36+ and MW of C36+ will be predicted using ML models.

| Detailed Set of Components | | | | | | | | | | | | | |
|---|---|---|---|---|---|---|---|---|---|---|---|---|---|
| CO2 | H2S | N2 | C1 | C2 | C3 | iC4 | nC4 | iC5 | nC5 | C6 | C7 | . . . | C36+ |

Sample 1
Sample 2
. . .
Sample i
. . .
Sample N_Samples

As a second example, some samples would have the following set of components and pseudo-components: CO2, H2S, N2, C1, C2, C3, iC4, nC4, iC5, nC5, C6, C7+. Mole fraction and MW of C12+, C20+ and C36+ will be predicted using ML models.

| Predefined Set of Components and Pseudo-Components | | | | | | | | | | | | |
|---|---|---|---|---|---|---|---|---|---|---|---|---|
| CO2 | H2S | N2 | C1 | C2 | C3 | C4 | C5 | C6 | C7+ | C12+ | C20+ | C36+ |

Sample 1
Sample 2
. . .
Sample i
. . .
Sample N_Samples

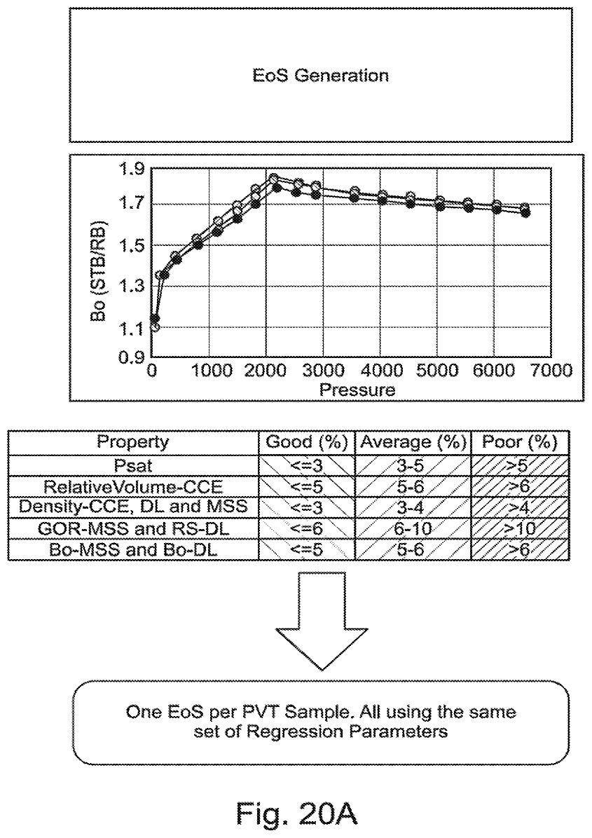

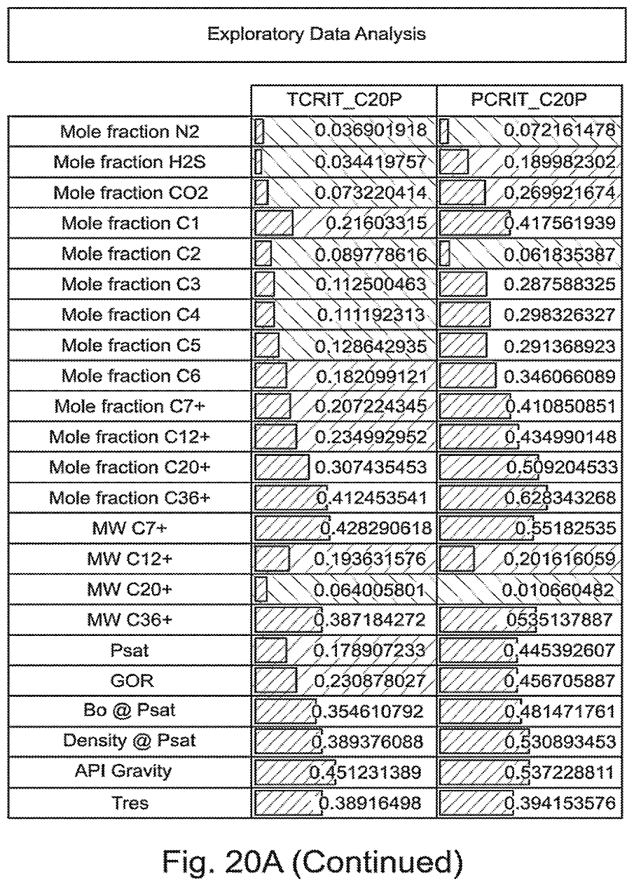

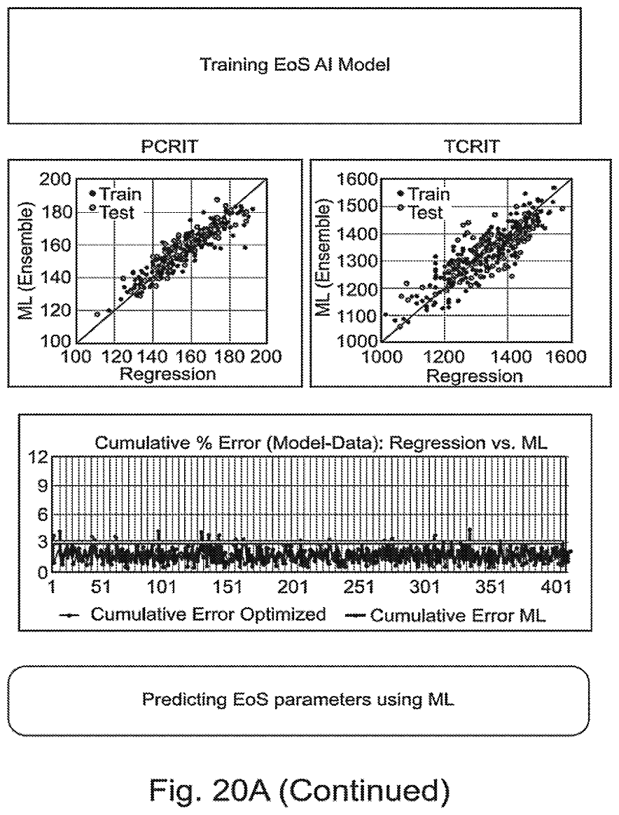

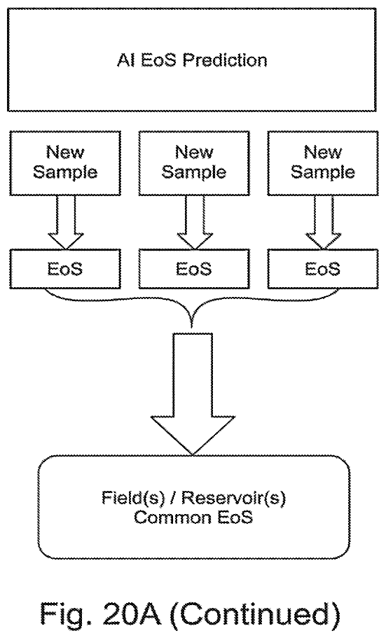

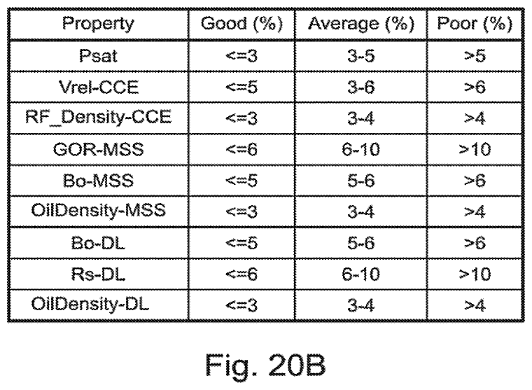

The step of lumping 202 into a predefined set of pseudo-components, in particular together with the step of delumping 201, are key to generate the multiple EoSs structured in the same format to enable building machine learning models. The step of delumping 201 refers to predicting a detailed composition of a sample. This delumping 201 is performed using the chain algorithm (see also description of FIG. 20A below). The mole fractions and molecular weights from the fluid C7+(or other tail) are determined. The delumping 201 gives the flexibility, based on the delumping 201 of SAMPLES into the detailed set of components to lump 202 the samples components into any pre-defined set of components and pseudo-components. Multiple lumping schemes as provided with the present invention, may then be used and following steps may be repeated using each of the lumping schemes. Optionally, if the delumping 201 results on a pre-defined set of components and pseudo-components, the step of lumping 202 may be omitted.

The following table shows an example for the delumping 201 and lumping 202. In the example shown, regression takes place on the lumped compositions with three pseud-components.

| | Mapping: | | Mapping: | |
|---|---|---|---|---|
| | Original- | | Detailed- | Lumped |
| Original | Detailed | Detailed | Final | (Example) |
| N2 | 1 | N2 | 1 | N2 |
| H2S | 2 | H2S | 2 | H2S |
| CO2 | 3 | CO2 | 3 | CO2 |
| C1 | 4 | C1 | 4 | C1 |
| C2 | 5 | C2 | 5 | C2 |
| C3 | 6 | C3 | 6 | C3 |
| iC4 | 7 | IC4 | 7 | C4 |
| nC4 | 8 | NC4 | 7 | C5 |
| neoC5 | 9 | IC5 | 8 | C6 |
| iC5 | 9 | NC5 | 8 | C7-C10 |
| nC5 | 10 | C6 | 9 | C11-C19 |
| C6 | 11 | C7 | 10 | C20+ |
| M-C-C5 | 12 | C8 | 10 | |
| Benzene | 12 | C9 | 10 | |
| Cyc-C6 | 12 | C10 | 10 | |
| C7 | 12 | C11 | 11 | |
| M-C-C6 | 13 | C12 | 11 | |
| Toluene | 13 | C13 | 11 | |
| C8 | 13 | C14 | 11 | |
| E-Benzene | 14 | C15 | 11 | |
| M/P- | 14 | C16 | 11 | |
| Xylene | | | | |
| O-Xylene | 14 | C17 | 11 | |
| C9 | 14 | C18 | 11 | |
| 1,2,4-TMB | 15 | C19 | 11 | |
| C10 | 15 | C20 | 12 | |
| C11 | 16 | C21 | 12 | |
| C12 | 17 | C22 | 12 | |
| C13 | 18 | C23 | 12 | |
| C14 | 19 | C24 | 12 | |
| C15 | 20 | C25 | 12 | |
| C16 | 21 | C26 | 12 | |
| C17 | 22 | C27 | 12 | |
| C18 | 23 | C28 | 12 | |
| C19 | 24 | C29 | 12 | |
| C20 | 25 | C30 | 12 | |
| C21 | 26 | C31 | 12 | |
| C22 | 27 | C32 | 12 | |
| C23 | 28 | C33 | 12 | |
| C24 | 29 | C34 | 12 | |
| C25 | 30 | C35 | 12 | |
| C26 | 31 | C36 | 12 | |
| C27 | 32 | | | |
| C28 | 33 | | | |
| C29 | 34 | | | |
| C30 | 35 | | | |
| C31 | 36 | | | |
| C32 | 37 | | | |
| C33 | 38 | | | |
| C34 | 39 | | | |
| C35 | 40 | | | |
| C36 | 41 | | | |

With respect to the step of generating 203 for the PVT samples on the PVT data set an EoS model using the same set of tuning parameters and thereby generating an EoS fluid model fingerprint for the hydrocarbon fluid samples, for each validated sample in the data base 301, an EoS model is automatically calibrated using the same set of tuning parameters. This results in a set of values of these tuning parameters, for each PVT sample, which will be considered as the EoS fluid model fingerprint for each sample. The following steps are provided:
Decide (user) on an EoS to be used (e.g. 3-Parameter Peng-Robinson EoS).
Decide (user) on EXPERIMENTS and corresponding weights.
Decide (user) on TUNING PARAMETERS.
Decide (user) on the data-model matching tolerance.
For every sample, Si, in SAMPLES, tune an EoS within the prescribed tolerance.
   For the results, a set of tuning parameters associated to the PVT sample Si, P1_Si, P2_Si, P3_Si, . . . , PN_Si, for every sample is provided.

| TUNING PARAMETERS Resulting from Step 3 | | | | | | |
|---|---|---|---|---|---|---|
| P1 | P2 | P3 | . . . | Pj | . . . | PN |

| | | | | | |
|---|---|---|---|---|---|
| Sample 1 | P1_1 | P2_1 | P3_1 | Pj_1 | PN_1 |
| Sample 2 | P1_2 | P2_2 | P3_2 | Pj_2 | PN_2 |
| . . . | | | | | |
| Sample i | P1_i | P2_i | P3_i | Pj_i | PN_i |
| . . . | | | | | |
| Sample N_Samples | P1_N_Samples | P2_N_Samples | P3_N_Samples | Pj_N_Samples | PN_N_Samples |

Further, with respect to the step of associating 204 properties of the hydrocarbon fluid samples with the generated EoS fluid model fingerprint, an association between samples properties and EoS fluid model fingerprint is build. This associating step 204 consists of training machine learning models to predict the EoS fluid model fingerprint for each sample and, therefore, EoS Models for any new PVT sample with the highest accuracy (see also description of FIG. 20A).

The use of machine learning to build models to predict P1, P2, P3, . . . , PN based on the PVT samples properties (Compositional and Black oil) has the following input:
a. Complete set of compositional and Black oil for all PVT samples resulting from the method 100 according to the first aspect of the invention.
b. Results of the EoS tuning for all SAMPLES from Step 3: P1_Si, P2_Si, P3_Si, . . . , PN_Si
   The use of machine learning to build models to predict P1, P2, P3, . . . , PN based on the PVT samples properties (Compositional and Black oil) has the following results: P1, P2, P3, . . . , PN that, when used in an EoS provide a full match between the laboratory results and EoS results; Fully machine learned EoS models.

Further, the associating step 204 of associating properties of the hydrocarbon fluid samples with the generated EoS fluid model fingerprint can optionally include clustering. Accordingly, multiple machine learning models can be trained on the different clusters as described with the method 100 according to the first aspect of the invention. Several validation steps and insights will be drawn by comparing the trends from clustering of original samples, EoS models, and from heatmaps applying EoS models on all samples; all aiming towards improving the accuracy of the predicted EoS Model.

The method 200 according the third aspect of the invention can further include the step of validating. The step of validating can be applied to new samples or samples that have not been used in the machine learning step. The step of validation can be processed as following:
1. A machine learned based EoS is build and loaded in a commercial PVT package
2. Results are compared with laboratory data.
3. Repeat Steps, as/if necessary, to optimize
   a. lumping scheme
   b. Set of tuning parameters.

FIG. 17B shows a flow chart describing a workflow for training of the ML algorithm and saving the models generated during the training of the ML algorithm. The trained clustered models (steps 902a, 902b) are saved (step 903) to be used later to predict a cluster ID for these new samples. After the selecting the ML algorithm and the testing of the ML algorithm on the unclassified data, the ML model is retrained on the whole dataset (both training data and test data, see above) for an improved prediction quality. All the trained ML models along with the clustering scheme are saved locally for future predictions.

FIG. 17C shows a workflow describing a method for clustering and saving the ML algorithms and ML models. The ML model is retrieved when a new sample (step 905) is evaluated based on the selected inputs and outputs. The cluster ID is predicted from the saved clustering scheme based on the cluster affiliation of every sample (steps 906, 907). The corresponding ML model is then utilized to predict the selected outputs for the selected inputs (steps 908, 909).

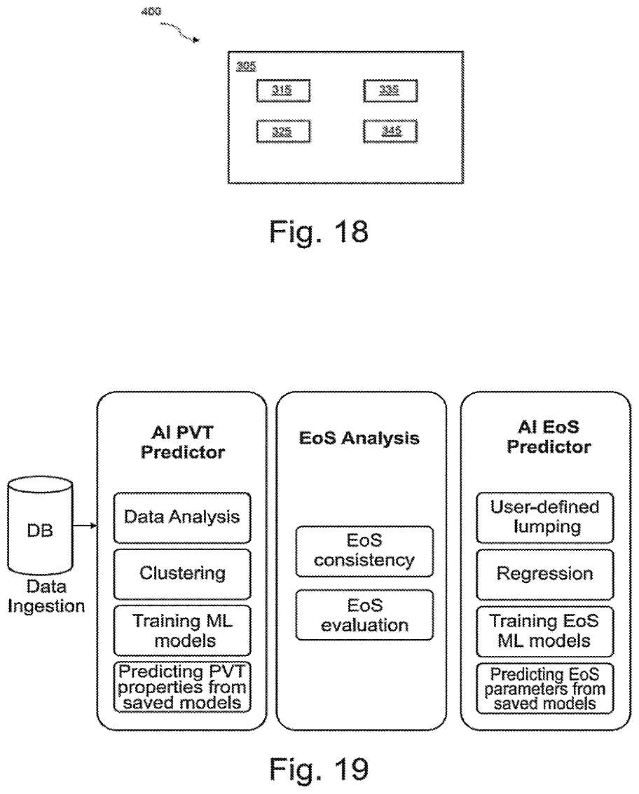

FIG. 18 is a schematic illustration of a system for generating equations of state (EoS) for a plurality of hydrocarbon fluids in accordance with a fourth aspect of the present invention.

The system 400 for generating equations of state (EoS) for a plurality of hydrocarbon fluids comprises a first module 315, a second module 325, a third module 335 and a fourth module 345. The first module 315 delumps the pressure-volume-temperature (PVT) data for the hydrocarbon fluid samples from a complete set of the PVT data to one of a set of detailed fluid components, or to a common set of components and pseudo-components. The second module 325 lumps the PVT data for hydrocarbon fluid sample into a pre-defined set of components and pre-defined set of pseudo-components to generate a plurality of equation of state (EoS) models. The third module 335 generates for the hydrocarbon fluid samples in a PVT data base an EoS model using a same set of tuning parameters and thereby generating an EoS fluid model fingerprint for the hydrocarbon fluid samples. The fourth module 345 associates properties of the hydrocarbon fluid samples with the generated EoS fluid model fingerprint.

FIG. 19 is an overview diagram of the hydrocarbon fluid properties and EoS prediction using machine learning subject of the present invention. As can be seen from FIG. 19, the system 300 comprises the AI PVT predictor, the EoS Analysis, and the AI EoS predictor.

FIG. 20A is an overview of the machine-learning based EoS Workflow in accordance with the present invention. In the below an illustration use of the machine-learning based EoS is presented. A data set of 853 hydrocarbon liquid samples is used. These hydrocarbon liquid samples have a complete data set of laboratory experiments: saturation pressure, constant composition expansion, differential liberation and multi-stage separator. Part of these hydrocarbon liquid samples have a detailed composition up to C36+. The other part of the hydrocarbon liquid samples has composition to different cut-off below C36+ Therefore, machine learning is used to complete the composition of these hydrocarbon liquid samples in order to have the entire set of hydrocarbon liquid samples with the same detailed components.

The workflow shown in FIG. 20A further elaborates the computer-implemented method 200 for generating the equations of state (EoS). As can be seen from FIG. 20A, the method 200 comprises preprocessing the samples using the chain algorithm to delump the samples using the ML algorithm. The composition of the delumped samples is then stored in the unified data structure. The method 200 further comprises loading the items of PVT data and the detailed composition of the items of PVT data into the EoS MODULE data structure. Based on this information, a user, for example, decides on the EoS to be used and on further laboratory experiments to be conducted. The user may further decide on the tuning parameters to be used, a data-model matching tolerance, and on the lumping scheme. The items of PVT data are then lumped in one set of components and pseudo-components. The EoS are then tuned within a prescribed tolerance according to the tuning parameters set by the user.

The ML algorithms are then used to predict to predict P1, P2, P3, . . . , PN based on the PVT sample properties (compositional properties and black oil properties). The inputs for the process are consists of compositional and black oil properties for all PVT samples. The results for the EoS tuning for the items in the PVT data are P1_Sᵢ, P2_Sᵢ, P3_Sᵢ, . . . , PN_Sᵢ. The output of the process is P1, P2, P3, . . . , PN. TheseTheseThis items of output data provide a full match between the laboratory results and the EoS results for the samples. The set of EoS tuning parameters resulting from the above steps is then validated and used by the EoS MODULE to model the PVT samples. The results are then compared with conventional regression-based results.

FIG. 20B shows a set of KPIs that can be used for a numerical evaluation of the EoS.

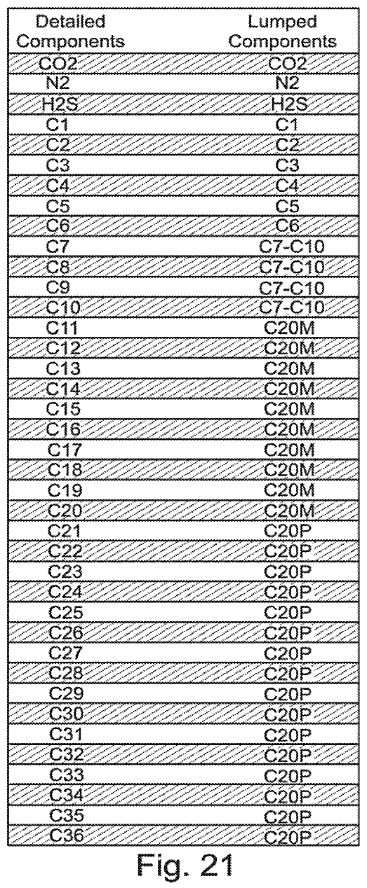

FIG. 21 illustrates the lumping scheme used in the process of creating the EoS model using regression. For the purpose of illustration, the detailed composition is lumped into 12 components and pseudo-components. As a result of this step, all 853 samples have the same set of lumped components and they are ready for regression. Different regression parameters where tested. As an illustration, here we present the results of regression using two parameters: PCRIT and TCRIT of C20P. The result of the regression on each of these samples is pair of PCRIT and TCRIT of C20P specific to that sample. The next step consists of using the results of regression in a machine learning model that predicts the EoS parameters; PCRIT and TCRIT of C20P in this specific case. Prior to doing so, Exploratory Data analysis (EDA) is used to select the set of parameters that have the highest impact on PCRIT and TCRIT of C20P.

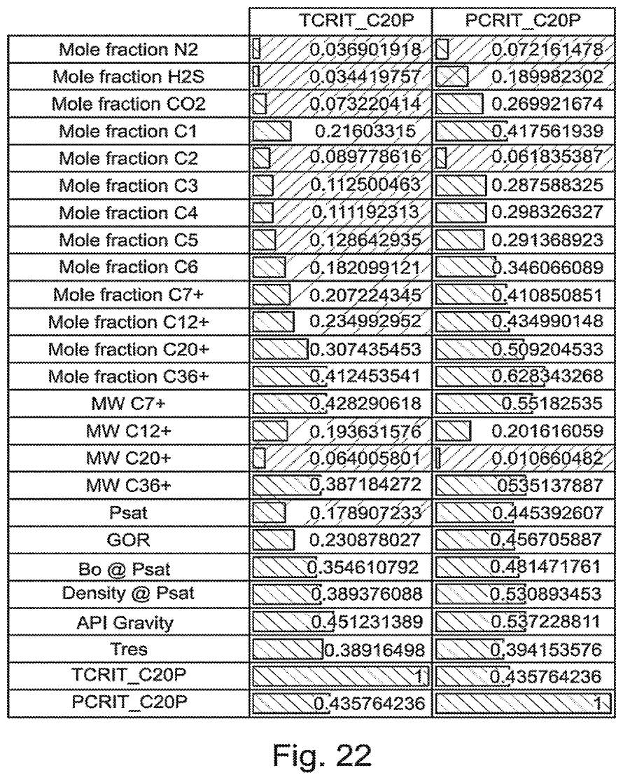

FIG. 22 is a heatmap resulting from Exploratory Data analysis (EDA) preceding machine learning to predict the EoS Parameters. TCRIT (C20P) and PCRIT (C20P) dependency on different BO and Compositional parameters. The results from EDA are the parameters to be used in machine learning. These are: Mole fraction of C1, C6, C7+, C12+, C20+, C36+, molecular weight of C7+, C12+, C36+, Psat, GOR, Bo@Psat, Density@Psat, API Gravity and Tres. Furthermore, chain algorithm is used: PCRIT of C20P is added to inputs to predict TCRIT of C20P.

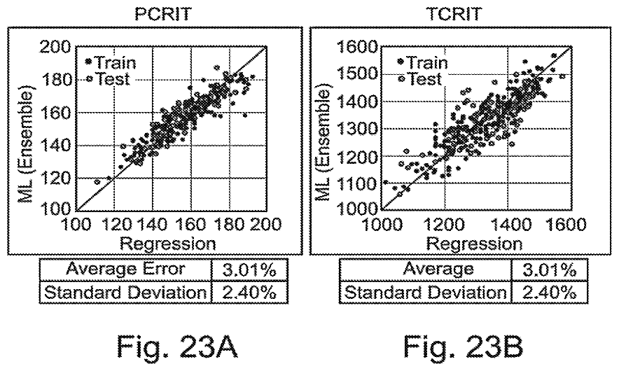

FIGS. 23A and 23B are an illustration of the predicted EoS parameters using machine learning when using Pcrit and Tcrit of C20P as regression parameters. The figure depicts Pcrit and Tcrit of C20P from machine learning vs. those obtained using conventional regression. The results of this step are machine learning predicted EoS parameters (PCRIT and TCRIT of C20P). These machine learning predicted parameters are then used in an EoS modeling tool (e.g. PVTi, PVTSim or any other tool) to predict the fluid properties and compare them with those obtained from laboratory measurement.

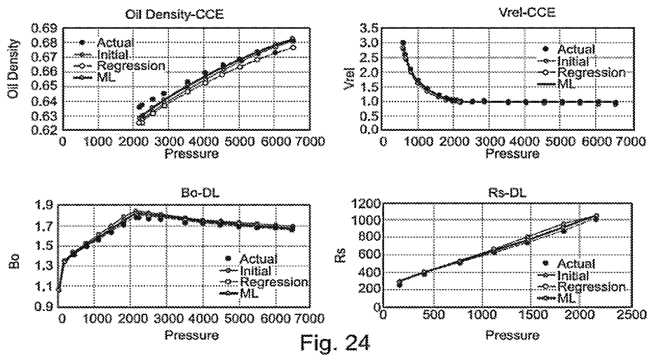

FIG. 24 illustrates example results of machine-learning based EoS—Sample 1, Field F1, Reservoir R1. The figure shows actual data, initial guess of EoS model without any regression, Conventional regression based EoS and ML based EoS.

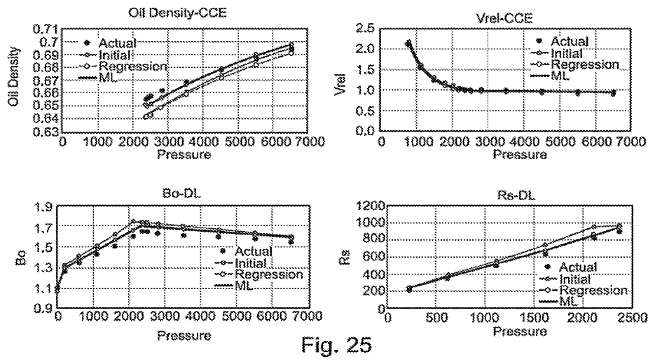

FIG. 25 illustrates other example results of machine-learning based EoS—Sample 2, Field F2, Reservoir R2. The figure shows actual data, initial guess of EoS model without any regression, Conventional regression based EoS and ML based EoS.

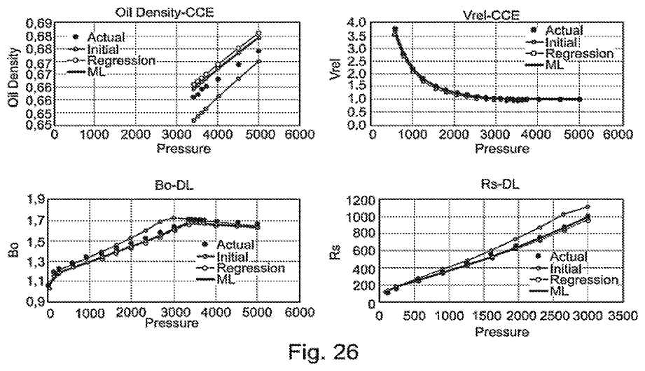

FIG. 26 illustrates other example results of machine-learning based EoS—Sample 3, Field F3, Reservoir R3. The figure shows actual data, initial guess of EoS model without any regression, Conventional regression based EoS and ML based EoS.

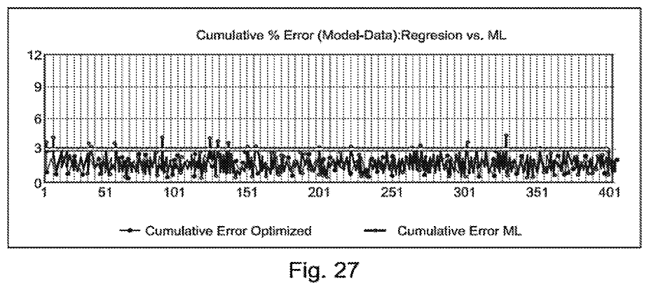

FIG. 27 illustrates machine-learning Based EoS—Error Analysis; Train+Test dataset. The EoS parameters resulting from ML are used in a PVT modeling tool to reproduce Laboratory experiments (CCE, DL and MSS). The figure shows the cumulative percentage error (EoS vs. Data) from conventional regression based EoS and machine learning based EoS.

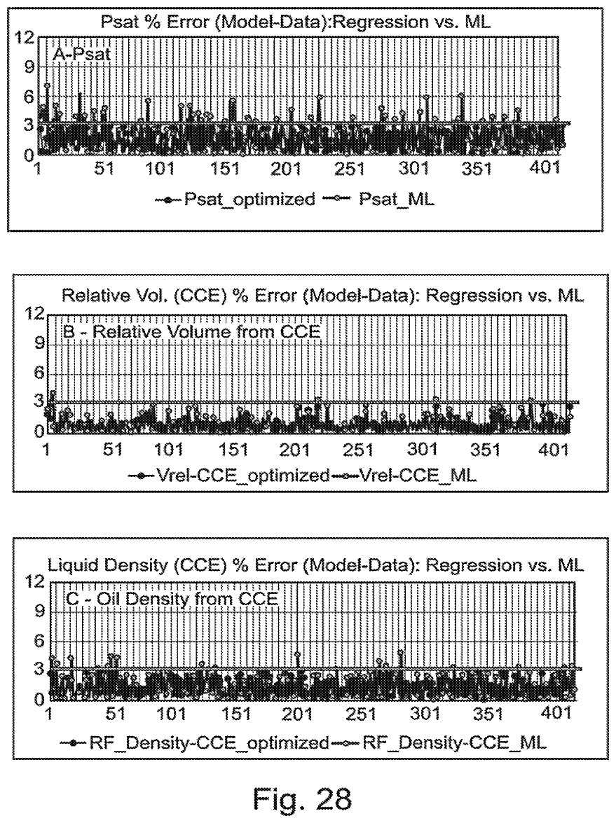

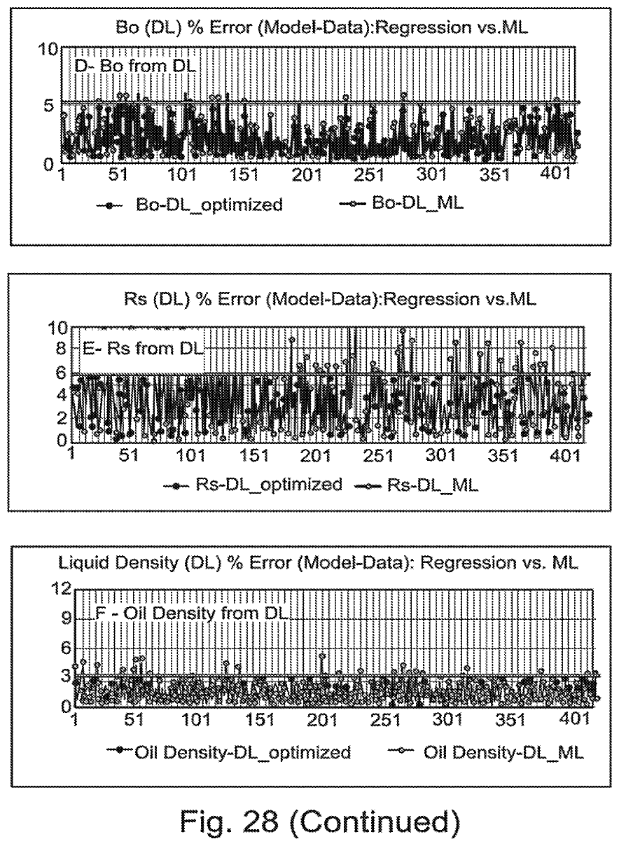

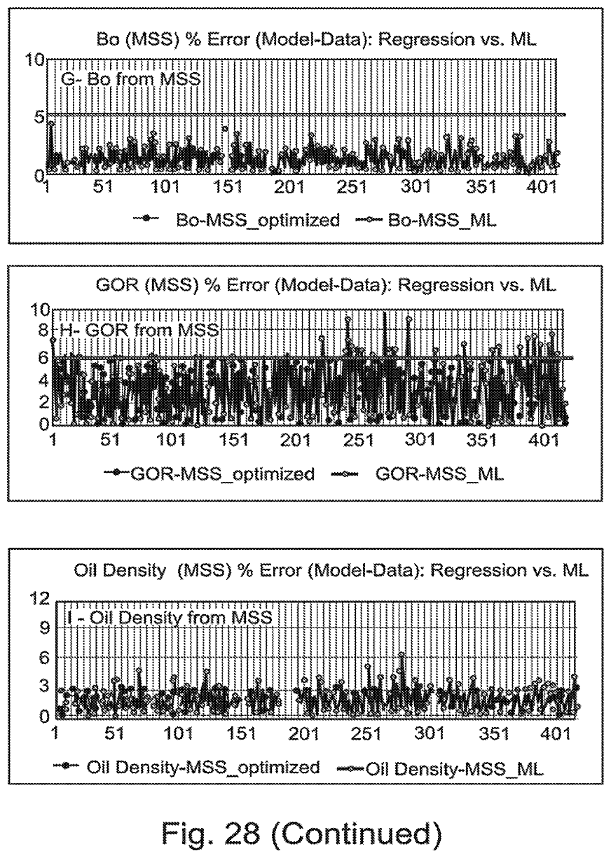

FIG. 28 illustrates machine-learning Based EoS—Error Analysis; Train+Test dataset. The EoS parameters resulting from ML are used in a PVT modeling tool to reproduce Laboratory experiments (CCE, DL and MSS). The figure shows the percentage error (EoS vs. Data) from conventional regression based EoS and machine learning based EoS for a selected set of fluid properties resulting from the above-mentioned experiments.

While only selected embodiments have been chosen to describe the present invention, it is apparent to the person skilled in the art from this disclosure that various changes and modifications can be made therein without deviating from the scope of the invention as defined in the attached claims.

1. A computer-implemented method for predicting hydrocarbon fluid properties using machine-learning-based models, the method comprising:
receiving an incomplete set of pressure-volume-temperature (PVT) data for hydrocarbon fluid samples from a PVT data base, wherein the incomplete set of PVT data comprises ones of black oil properties and compositional properties;
reading the incomplete set of PVT data by a reader module;
transforming the incomplete set of PVT data into a unified data structure by the reader module, wherein the unified data structure is used for storing items of data input from different sources in a unified way;
selecting items of the PVT data from the transformed incomplete set of PVT data by the reader module using exploratory data analysis (EDA);
processing the selected items of the transformed incomplete set of PVT data by a correlating module to identify a plurality of correlations in the selected items of the transformed incomplete set of PVT data based on one or more of the fluid properties of the hydrocarbon fluid samples;
clustering, using of at least one of a plurality of clustering schemes, the selected items of the transformed incomplete set of PVT data into a plurality of clusters by a clustering module; and
performing machine learning by a machine learning module on ones of the plurality of clusters to predict missing fluid properties in the incomplete set of PVT data and thus to obtain a complete set of PVT data, wherein the predicted complete set of PVT data comprises the black oil properties and compositional properties of the incomplete set of PVT data and further comprises the predicted items of data for the ones of the black oil properties and predicted compositional properties.

2. The computer-implemented method according to claim 1, wherein the step of performing machine learning by the machine learning module further comprises the step of predicting of fluid properties for the incomplete sets of PVT data for the hydrocarbon fluid samples.

3. The computer-implemented method according to claim 1, further comprising the step of plotting the machine learning predictions by the machine learning module.

4. The computer-implemented method according to claim 1, further comprising the step of comparing the identified plurality of correlation results with the machine learning predictions.

5. The computer-implemented method according to claim 1, wherein the clustering further comprises the step of completing fluid composition including C12+, C20+ and C36+ mole fraction and molecular weight and/or completing black oil properties, including, in the following order: solution gas oil ratio (GOR), BO@Psat, and saturation pressure (Psat).

6. A computer-implemented method for generating equations of state (EoS) for a plurality of hydrocarbon fluids from a predicted complete set of PVT data, the method comprising:
delumping pressure-volume-temperature (PVT) data for hydrocarbon fluid samples from the predicted complete set of PVT data to one of a set of detailed fluid components, or to a common set of components and pseudo-components;
lumping the PVT data from the predicted complete set of PVT data into a pre-defined set of components and pre-defined set of pseudo-components to generate a plurality of equation of state (EoS) models;
generating for the PVT samples on the PVT data set an EoS model using a same set of tuning parameters and thereby generating an EoS fluid model fingerprint for the hydrocarbon fluid samples; and
associating properties of the hydrocarbon fluid samples with the generated EoS fluid model fingerprint.

7. The computer-implemented method according to claim 6, further comprising training machine learning models to predict an EoS fluid model fingerprint for new hydrocarbon fluid samples.

8. The computer-implemented method according to claim 1, wherein the incomplete set of PVT data for hydrocarbon fluid samples comprises black oil properties and compositional properties, wherein the black oil properties comprise at least one of reservoir temperature, solution gas-oil ratio, oil API gravity, gas gravity, dead oil viscosity, saturation pressure, saturated bubble point oil formation factor at saturation pressure, fluid density at reservoir conditions, fluid compressibility at reservoir conditions, viscosity at reservoir conditions, fluid density at reservoir conditions or any other black oil property, and wherein the compositional properties comprise at least one of mole fractions of the components, in particular N2, H2S, CO2, C1, C2, C3, C4, C5, C7, C8, and pseud-components, in particular C7+, C12+, C20+ and C36+ or any other pseudo-component as well as the molecular weight of the pseudo-components.

9. The computer-implemented method according to claim 6, wherein the step of associating comprises the method step of clustering the selected items of the PVT data into a plurality of clusters for performing machine learning on every one of the plurality of clusters.

10. The computer-implemented method according to claim 9, further comprising the step of comparing the results from clustering of the selected items of the PVT data with the plurality of equations of state (EoS) models and with heatmaps applying EoS models on the selected items of the PVT data.

11. The computer-implemented method according to claim 1, wherein the clustering of the selected items of the PVT data includes the step of identifying of clusters to which PVT data belong.

12. A system for predicting hydrocarbon fluid properties using machine-learning-based models, the system comprising:
a pressure-volume-temperature (PVT) data base providing an incomplete set of pressure-volume-temperature (PVT) data for hydrocarbon fluid samples, wherein the incomplete set of PVT data comprises ones of black oil properties and compositional properties;
a reader module for reading in the incomplete set of PVT data, wherein the reader module is configured to transform the incomplete set of PVT data into a unified data structure, and wherein the unified data structure is used for storing items of data input from different sources in a unified way, and wherein the reader module is configured to select items of the PVT data from the incomplete set of PVT data using exploratory data analysis (EDA);
a correlating module for processing the selected items of the transformed incomplete set of PVT data to identify a plurality of correlations in the selected items of the PVT data based on one or more of the fluid properties of the hydrocarbon fluid samples;
a clustering module for clustering, using of at least one of a plurality of clustering schemes, the selected items of the transformed incomplete set of PVT data into a plurality of clusters; and
a machine learning module performing machine learning on ones of the plurality of clusters to predict missing fluid properties in the incomplete set of PVT data and thus to obtain a complete set of PVT data, wherein the predicted complete set of PVT data comprises the black oil properties and compositional properties of the incomplete set of PVT data and further comprises the predicted items of data for the ones of the black oil properties and predicted compositional properties.

13. A system for generating equations of state (EoS) for a plurality of hydrocarbon fluids from a predicted complete set of PVT data, the system comprising
a first module for delumping pressure-volume-temperature (PVT) data for hydrocarbon fluid samples from the predicted complete set of PVT data to one of a set of detailed fluid components, or to a common set of components and pseudo-components,
a second module for lumping the PVT data for hydrocarbon fluid sample into a pre-defined set of components and pre-defined set of pseudo-components to generate a plurality of equation of state (EoS) models,
a third module for generating for the hydrocarbon fluid samples in a PVT data base an EoS model using a same set of tuning parameters and thereby generating an EoS fluid model fingerprint for the hydrocarbon fluid samples; and
a fourth module configured to associate properties of the hydrocarbon fluid samples with the generated EoS fluid model fingerprint.
